(19) United States
(12) Patent Application Publication
(10) Pub. No.: US 2021/0025902 A1
(43) Pub. Date: Jan. 28, 2021

(54) BIOMARKERS FOR UROLOGIC CHRONIC PELVIC PAIN SYNDROME

(71) Applicant: CEDARS-SINAI MEDICAL CENTER, Los Angeles, CA (US)

(72) Inventors: Jennifer ANGER, Manhattan Beach, CA (US); Anne Lenore ACKERMAN, Los Angeles, CA (US); Jennifer VAN EYK, Los Angeles, CA (US); Qin FU, Beverly Hills, CA (US); Weston SPIVIA, West Hollywood, CA (US)

(73) Assignee: Cedars-Sinai Medical Center, Los Angeles, CA (US)

(21) Appl. No.: 16/975,482

(22) PCT Filed: Feb. 26, 2019
(86) PCT No.: PCT/US2019/019644

§ 371 Date: Aug. 25, 2020

Related U.S. Application Data
(60) Provisional application No. 62/635,375, filed on Feb. 26, 2018.

(51) Int. Cl. G01N 33/68; G16H 50/30; G16H 50/20; G16H 10/40

(52) U.S. Cl. CPC G01N 33/6893; G16H 10/40; G16H 50/20; G16H 50/30

(57) ABSTRACT

Described herein are methods for determining the status of Urologic Chronic Pelvic Pain Syndrome (UCPPS) in a subject and/or treating urologic chronic pelvic pain syndrome (UCPPS).

### CROSS-REFERENCE TO RELATED APPLICATION

This application claims the benefit under 35 U.S.C § 119(e) of U.S. Provisional Patent Application No. 62/635,375 filed on Feb. 26, 2018, which is incorporated herein by reference in its entirety.

### STATEMENT REGARDING FEDERALLY SPONSORED RESEARCH OR DEVELOPMENT

This invention was made with government support under Grant No. DK103260 awarded by National Institutes of Health. The government has certain rights in the invention.

### FIELD OF THE INVENTION

Described herein are methods for determining the status of Urologic Chronic Pelvic Pain Syndrome (UCPPS) in a subject and/or treating urologic chronic pelvic pain syndrome (UCPPS).

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Many people suffer from or are at risk of developing urologic chronic pelvic pain syndrome (UCPPS). As such there is a need for methods for identification/discovery of protein biomarkers for urologic chronic pelvic pain syndrome (UCPPS), and for determining the status of Urologic Chronic Pelvic Pain Syndrome (UCPPS) in a subject and/or treating urologic chronic pelvic pain syndrome (UCPPS).

### SUMMARY OF THE INVENTION

The following embodiments and aspects thereof are described and illustrated in conjunction with systems, articles of manufacture, compositions and methods which are meant to be exemplary and illustrative, not limiting in scope.

In various embodiments, the present invention provides a method for determining status of Urologic Chronic Pelvic Pain Syndrome (UCPPS) in a subject, comprising: obtaining a sample from the subject, wherein the subject has Urologic Chronic Pelvic Pain Syndrome (UCPPS), and wherein the sample is selected from blood, serum, plasma, urine, and a combination thereof; detecting an amount of at least one biomarker in the sample, wherein the biomarker is a protein selected from A1AT, APOC2, APOA4, ITIH2, VTNC, APOC1, CO4A, GEL, IC1, AACT, APOA1, A1BG, APOE, C1R, IGHG3, A1AG1, ALBU, VTDB, KLKB1, and a combination thereof; and comparing the amount of the at least one biomarker in the sample from the subject to an amount of the at least one biomarker in a reference to determine the status of Urologic Chronic Pelvic Pain Syndrome (UCPPS) in a subject.

In some embodiments, the reference is selected from: (i) a baseline value for the amount of the biomarker, wherein the baseline value is from at least one sample obtained from the subject at an earlier point in time; (ii) a reference sample from a control subject, wherein the control subject does not have Urologic Chronic Pelvic Pain Syndrome (UCPPS); (iii) a reference sample from a control subject, wherein the control subject has Urologic Chronic Pelvic Pain Syndrome (UCPPS); (iv) a range of reference values for the amount of the biomarker, wherein the range of reference values is from at least one reference sample obtained from at least one healthy subject; (v) a range of reference values for the amount of the biomarker, wherein the range of reference values is from at least one sample obtained from the subject at an earlier point in time; and (vi) a range of reference values for the amount of the biomarker, wherein the range of reference values is from at least one reference sample obtained from at least one reference subject, wherein the reference subject has Urologic Chronic Pelvic Pain Syndrome (UCPPS).

In some embodiments, an increase in the amount of the at least one biomarker in the sample from the subject relative to the amount of the at least one biomarker in the reference is used to determine a Rand Interstitial Cystitis Epidemiology (RICE) subtype in the subject, wherein the biomarker is selected from A1AT, ITIH2, VTNC, APOC1, IC1, AACT, APOA1, A1BG, APOE, C1R, IGHG3, A1AG1, ALBU, VTDB, and KLKB1.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the biomarker is selected from A1AT, APOC2, APOA4, ITIH2, and VTNC, and wherein severity of urinary symptoms of Urologic Chronic Pelvic Pain Syndrome (UCPPS) in the subject is scored according to the urinary severity index.

In some embodiments, the baseline value for the amount of the biomarker in the sample from the subject is dependent on a characteristic of the subject, wherein the biomarker is selected from A1AT, ITIH2, and VTNC, and wherein the characteristic of the subject is selected from age, Rand Interstitial Cystitis Epidemiology (RICE) subtype, urinary severity index score, and a combination thereof.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the baseline value for the amount of the biomarker in the sample from the subject is dependent on a characteristic of the subject, wherein the biomarker is selected from A1AT, ITIH2, VTNC, AACT, A1BG, IGHG3, A1AG1, ALBU, and VTDB, and wherein the characteristic of the subject is selected from age, sex, and a combination thereof.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the method further comprises using the amount of the biomarker in the sample from the subject to distinguish between (a) a diagnosis of pelvic pain in the subject, and (b) a diagnosis of pelvic pain and beyond in the subject, wherein the biomarker is selected from APOC1 and CO4A.

In some embodiments, the biomarker is APOC1, and wherein the amount of the biomarker in the sample from the subject is dependent on a characteristic of the subject, wherein the characteristic of the subject is selected from sex, Rand Interstitial Cystitis Epidemiology (RICE) subtype, and a combination thereof.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the method further comprises using the amount of the biomarker in the sample from the subject to assist in diagnosing flare status of Urologic Chronic Pelvic Pain Syndrome (UCPPS) in the subject, wherein the biomarker is GELS.

In some embodiments, the amount of the biomarker in the sample from the subject is increased relative to the amount of the biomarker in the reference, wherein the biomarker is selected from C1R and A1AT, and wherein the reference is a reference sample from a control subject, wherein the control subject does not have Urologic Chronic Pelvic Pain Syndrome (UCPPS).

In some embodiments, the biomarker is A1AT, and wherein the amount of the biomarker in the sample from the subject is dependent on a characteristic of the subject, wherein the characteristic of the subject is selected from age, sex, urinary severity index score, and a combination thereof.

In some embodiments, the biomarker is C1R, and wherein the amount of the biomarker in the sample from the subject is dependent on a characteristic of the subject, wherein the characteristic of the subject is selected from age, sex, and a combination thereof.

In some embodiments, the biomarker is selected from APOA1, A1BG, APOE, A1AT, IGHG3, ALBU, VTDB, VTNC, and ITIH2, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) both painful filling and painful urgency, and (b) neither painful filling nor painful urgency.

In some embodiments, the biomarker is selected from AACT, A1BG, C1R, A1AT, IGHG3, VTDB, VTNC, IC1, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) painful urgency, and (b) neither painful filling nor painful urgency.

In some embodiments, the biomarker is A1AT, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) painful urgency, and (b) both painful filling and painful urgency.

In some embodiments, the biomarker is selected from A1AT, A1AG1, and VTNC, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) painful filling, and (b) painful urgency.

In some embodiments, the amount of the biomarker in the sample from the subject is dependent on a characteristic of the subject, wherein the characteristic of the subject is sex, and wherein the biomarker is selected from IC1, AACT, APOC1, and KLKB1.

In some embodiments, the amount of the biomarker in the sample from the subject is dependent on a characteristic of the subject, wherein the characteristic of the subject is age, and wherein the biomarker is selected from A1AG1 and ALBU.

In some embodiments, the amount of the biomarker in the sample from the subject is dependent on a characteristic of the subject, wherein the characteristic of the subject is selected from sex, age, and a combination thereof, and wherein the biomarker is selected from A1BG, IGHG3, and VTDB.

In some embodiments, the amount of the biomarker in the sample from the subject is dependent on a characteristic of the subject, wherein the characteristic of the subject is selected from age, urinary severity index score, and a combination thereof, and wherein the biomarker is ITIH2.

In some embodiments, the amount of the biomarker in the sample from the subject is dependent on a characteristic of the subject, wherein the characteristic of the subject is selected from age, sex, urinary severity index score, and a combination thereof, and wherein the biomarker is selected from A1AT and VTNC.

In some embodiments, the method further comprises administering a treatment for Urologic Chronic Pelvic Pain Syndrome (UCPPS) to the subject.

In some embodiments, the method further comprises administering a treatment for a symptom of Urologic Chronic Pelvic Pain Syndrome (UCPPS) to the subject.

In some embodiments, the amount of the biomarker is detected by mass spectrometry, an immunoassay, or a capture and detection assay.

In some embodiments, the mass spectrometry comprises operating a mass spectrometer.

In some embodiments, the immunoassay comprises performing the immunoassay.

In some embodiments, the capture and detection assay comprises performing the capture and detection assay.

In some embodiments, the biomarker is an isoform, single-nucleotide polymorphism, or fragment of the protein.

In some embodiments, the protein comprises a post-translational modification.

In some embodiments, the subject is human.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1B depicts in accordance with various embodiments of the invention, levels of APOC1 and CO4A can distinguish whether a subject is diagnosed to have pelvic pain (PP) only as compared to pelvic pain (PP) and beyond. The Y-axis values show area under the curve.

FIG. 2A-FIG. 2B depicts in accordance with various embodiments of the invention, level of GELS can be used to assist in the diagnosis of subject Flare status within Urologic Chronic Pelvic Pain Syndrome. X-axis values mean Healthy control, Patient with Flare, and Patient without Flare. Y-axis shows the area under the curve. 1T denotes the data came from a blend of healthy and disease cohorts sampled once. 2T denotes data came from disease cohort patient samples twice over 13 months. (n.s. stands for “not significant”).

FIG. 3A-FIG. 3M depicts in accordance with various embodiments of the invention, level of listed markers can assist in determining the status of a patient within the Rand Interstitial Cystitis Epidemiology (RICE) criteria consisting of the IC subtypes of painful filling, painful urgency, neither painful filling nor painful urgency, and both painful filling and painful urgency. IT denotes the data came from a blend of healthy and disease cohorts sampled once. 2T denotes data came from disease cohort patient samples twice over 13 months. In FIG. 3B, A1AT can also distinguish between healthy control and patients. In FIG. 3J, C1R can also distinguish between healthy control and patients.

FIG. 4A-FIG. 4G depicts in accordance with various embodiments of the invention, level of listed marker is correlated with the urinary severity index score. The “Urinary severity index” is an established index used to score patient symptom severity. 1T denotes the data came from a blend of healthy and disease cohorts sampled once. 2T denotes data came from disease cohort patient samples twice over 13 months.

FIG. 5A-FIG. 5C depicts in accordance with various embodiments of the invention, levels of markers that are significantly different based on sex. Y-axis is area under the curve. 1T denotes the data came from a blend of healthy and disease cohorts sampled once. 2T denotes data came from disease cohort patient samples twice over 13 months.

FIG. 6A-FIG. 6J depicts various embodiments of the invention, proteins with an interaction with Sex and RICE subtype. These proteins may have the same average protein abundance between the sexes, but there are differences in abundance of the protein within the RICE Subtypes. Plotted on the y-axis is the mean Area per classification of RICE Subtype. The dashed line is for Females, while the solid line is for males. 1T denotes the data came from a blend of healthy and disease cohorts sampled once. 2T denotes data came from disease cohort patient samples twice over 13 months.

FIG. 7A-FIG. 7P depicts various embodiments of the invention, proteins that change with Age. Level of listed marker is correlated with the Age in years. 1T denotes the data came from a blend of healthy and disease cohorts sampled once. 2T denotes data came from disease cohort patient samples twice over 13 months.

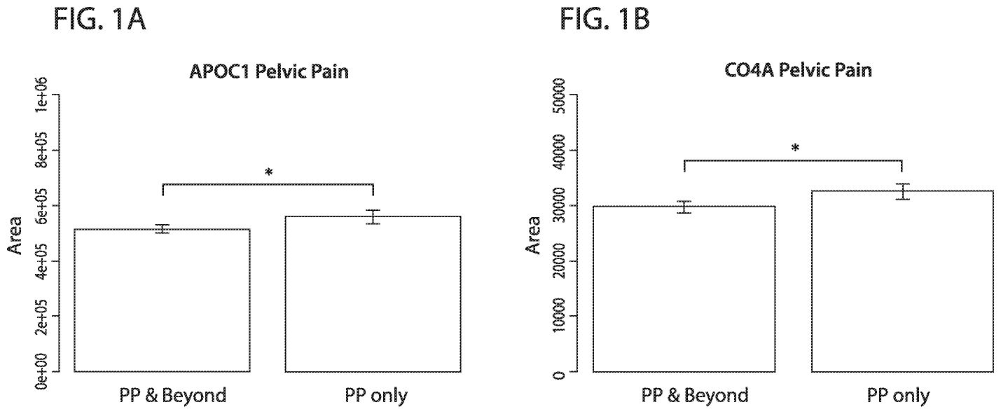

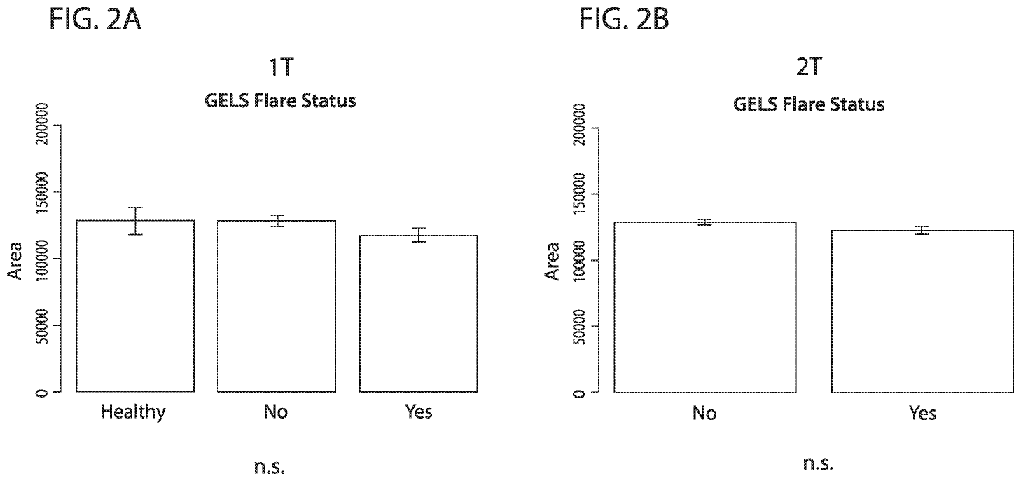

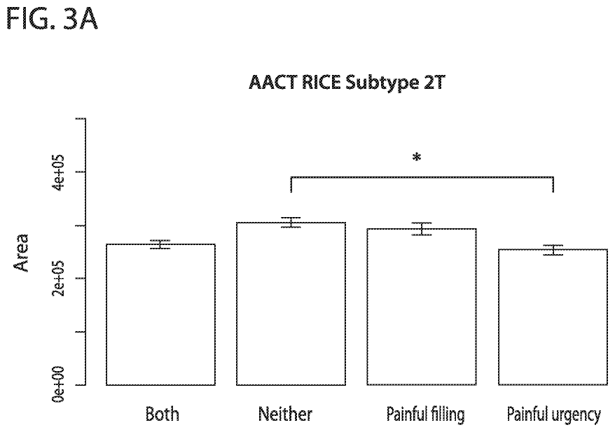

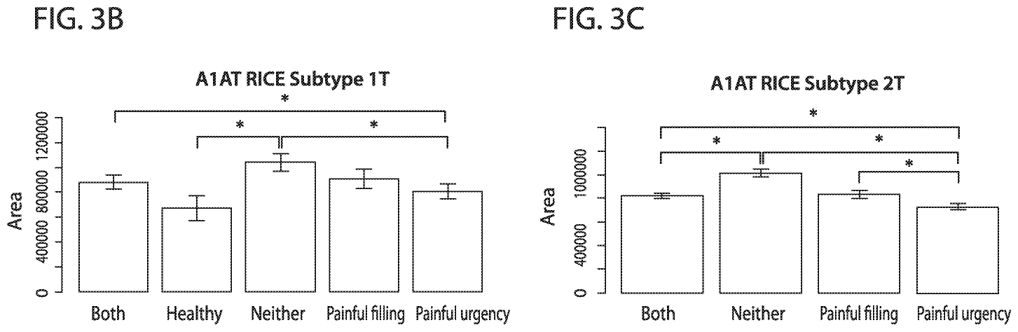

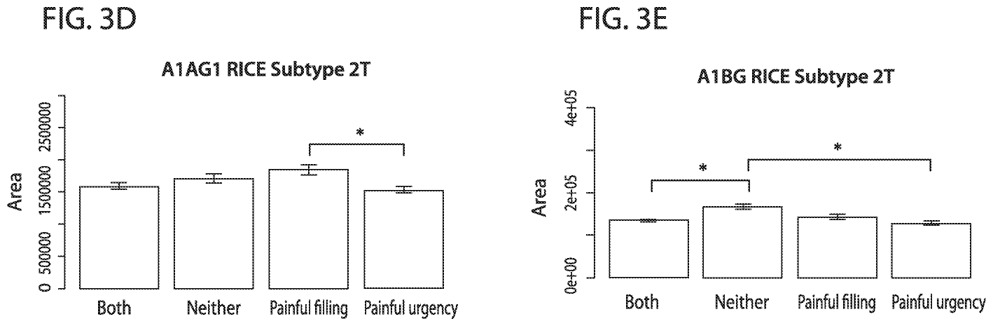

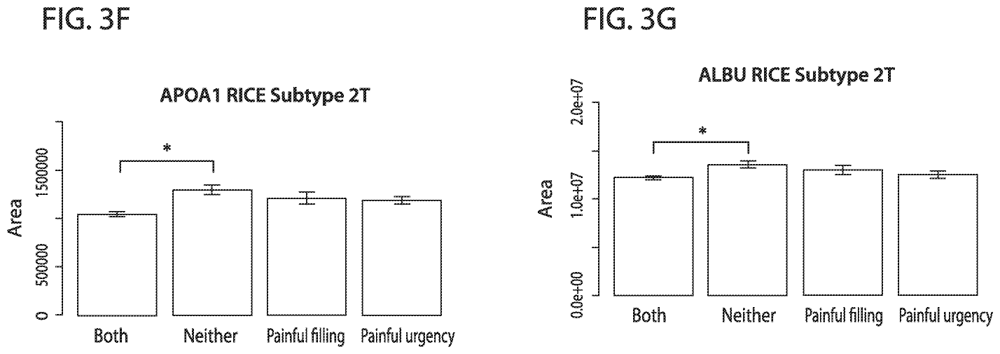

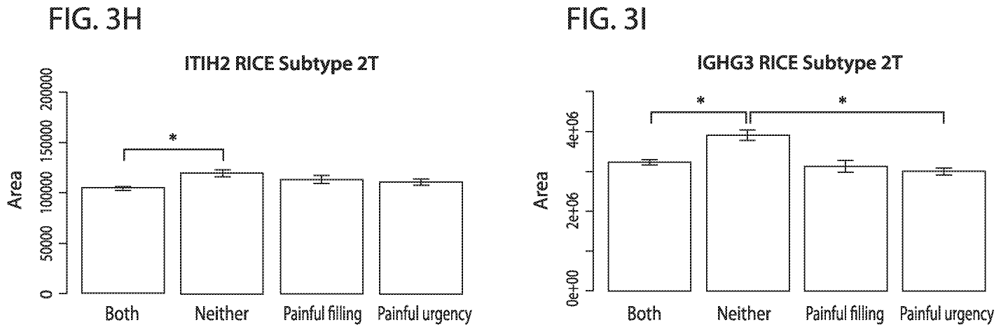

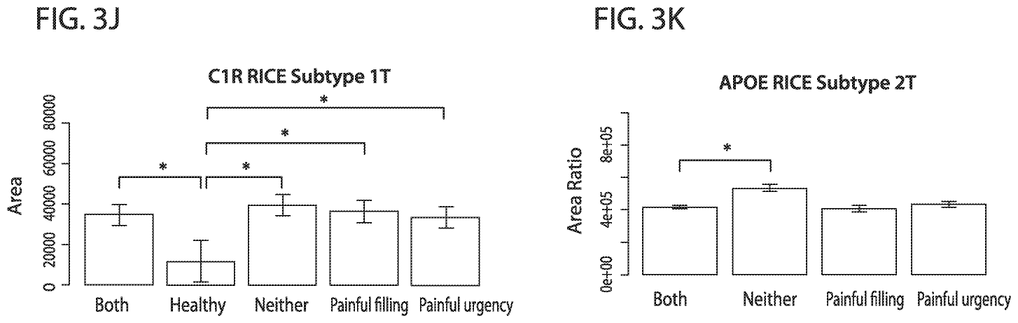

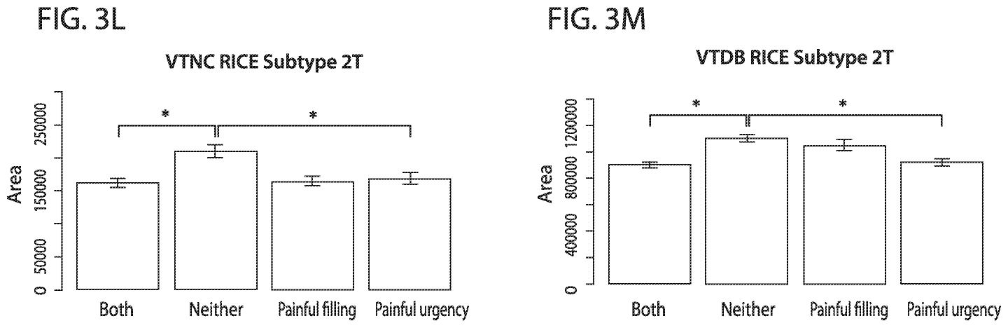

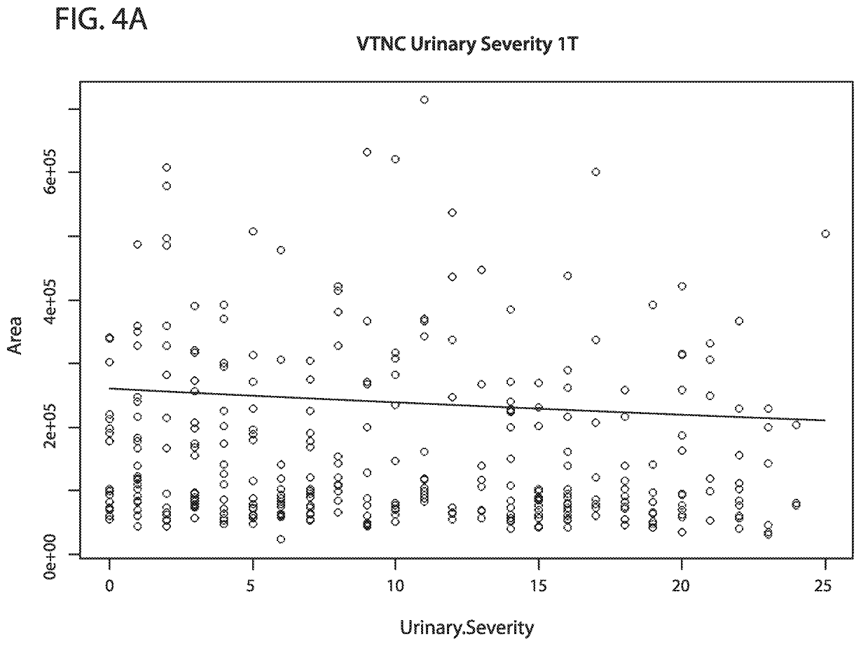

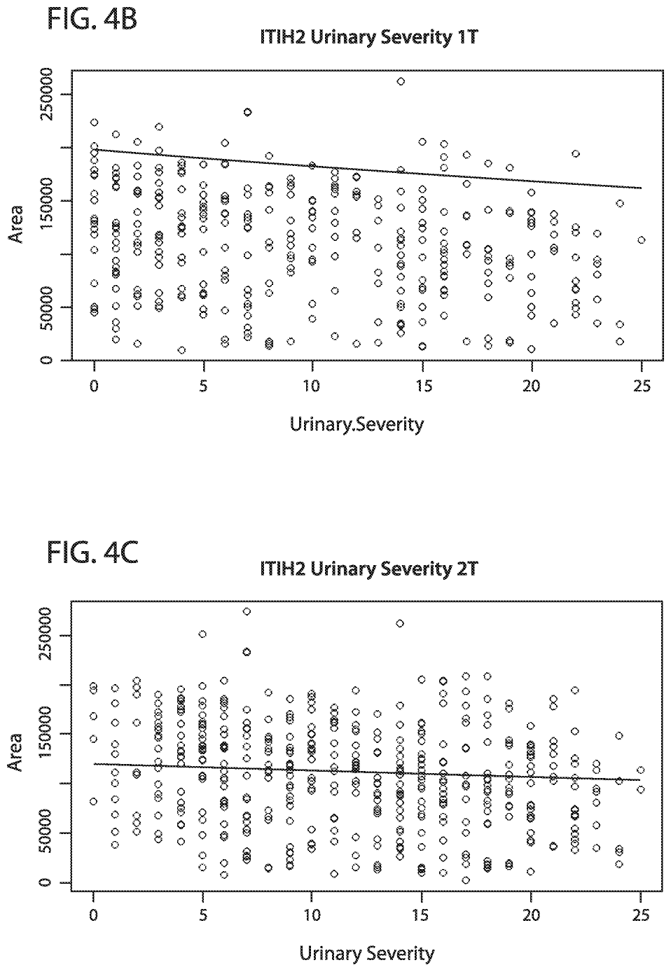

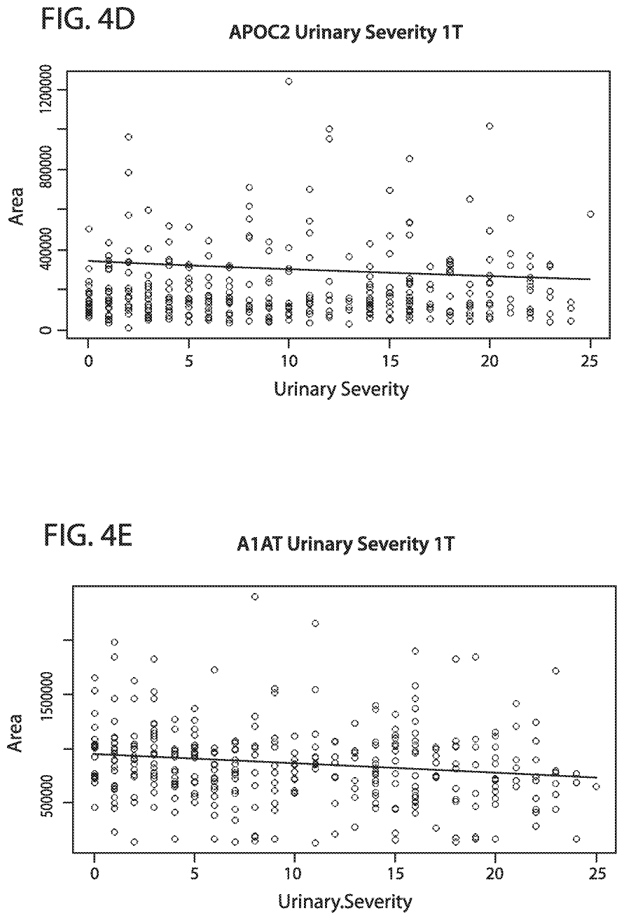

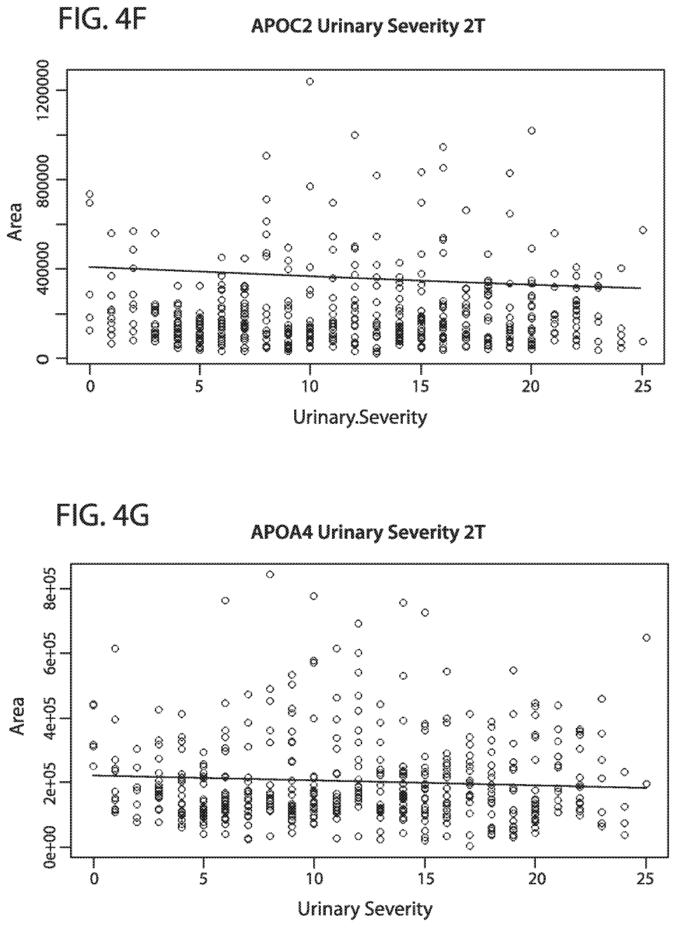

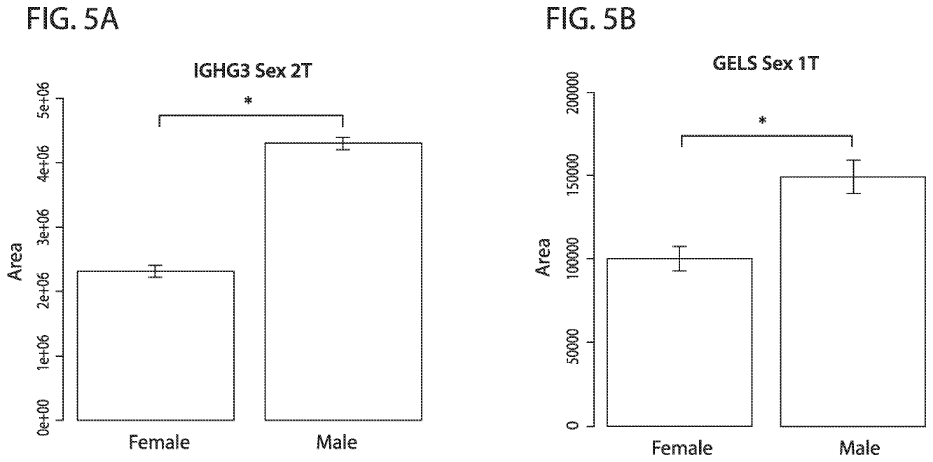

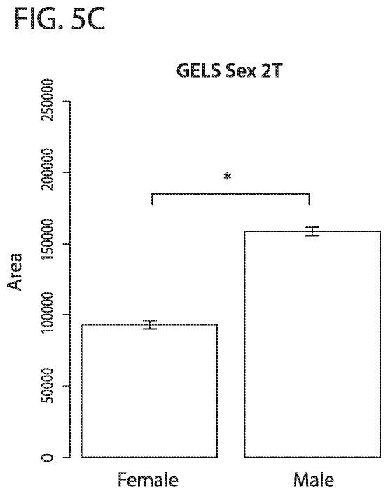

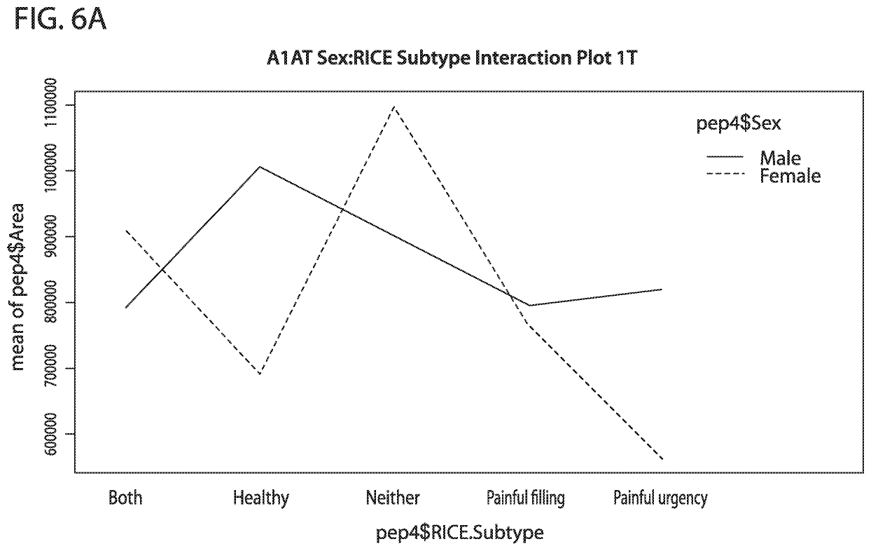

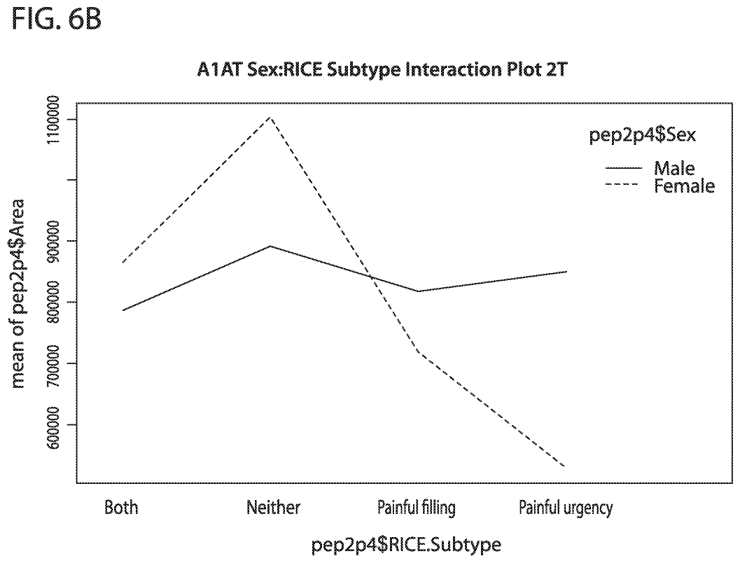

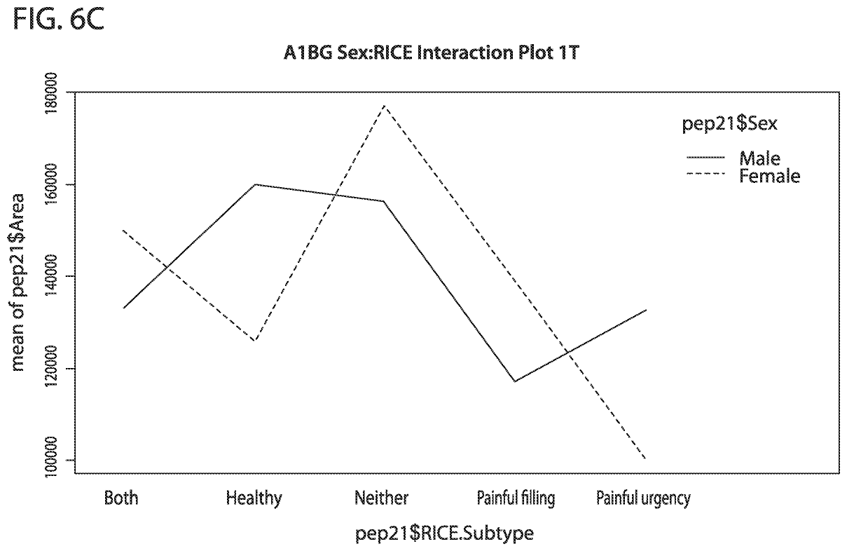

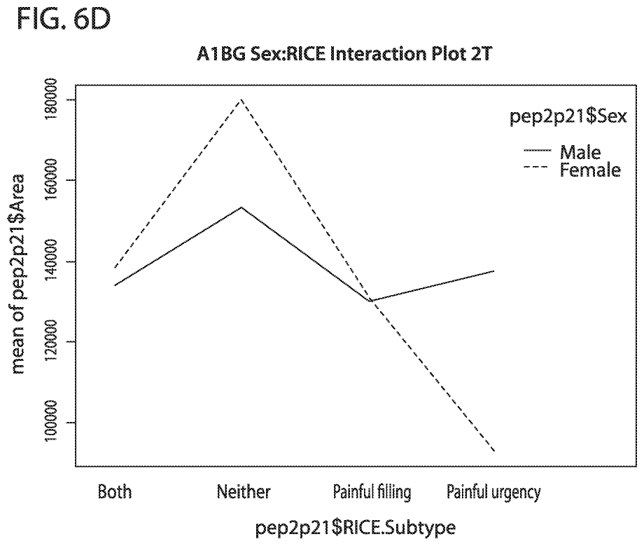

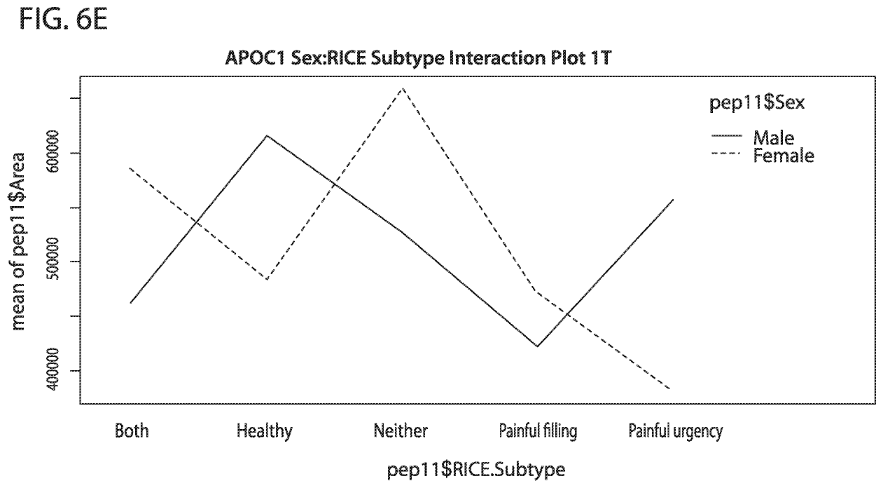

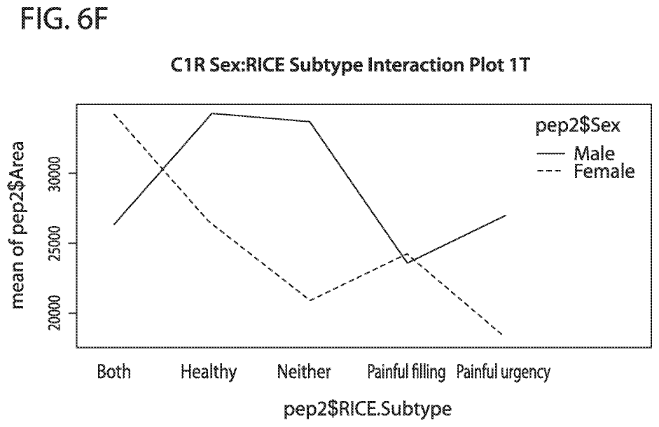

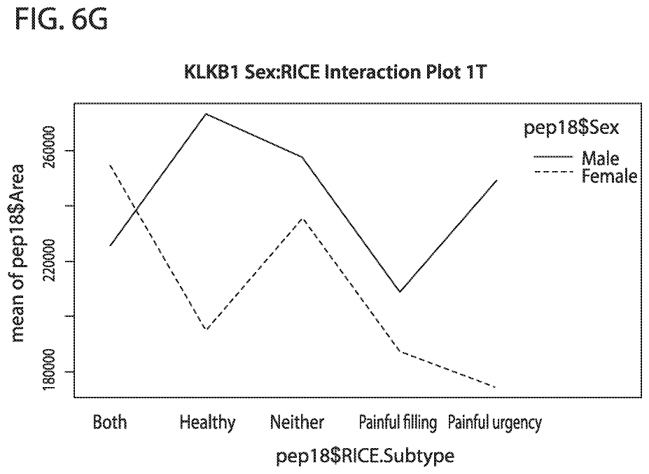

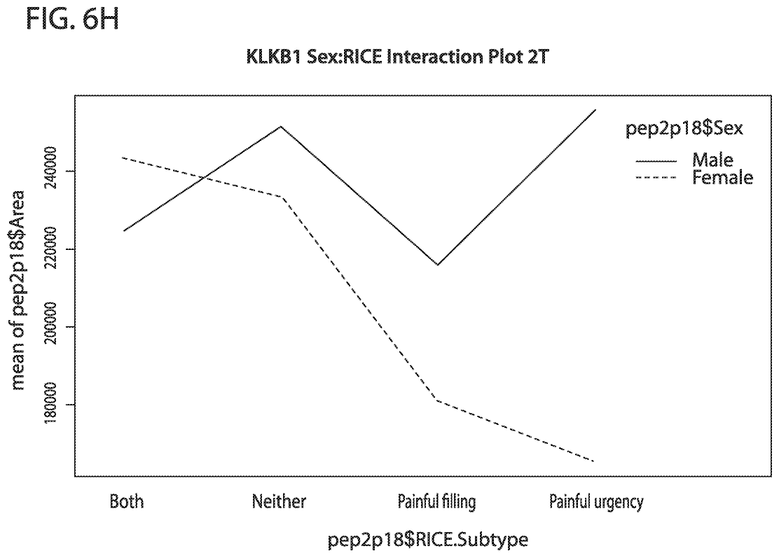

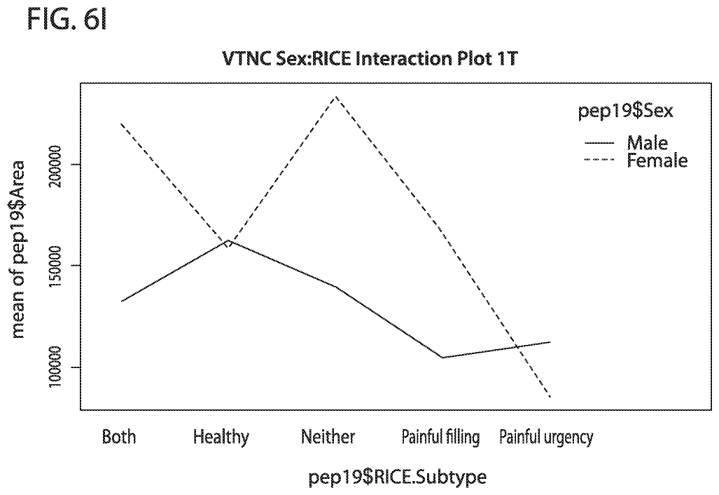

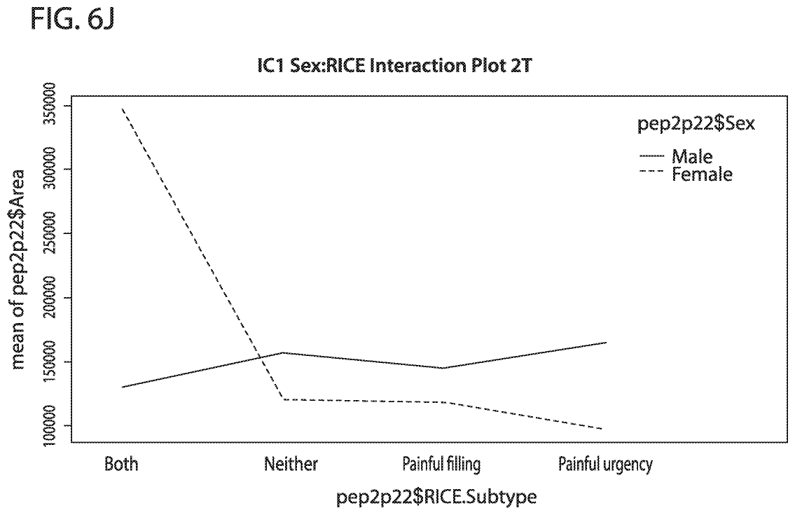

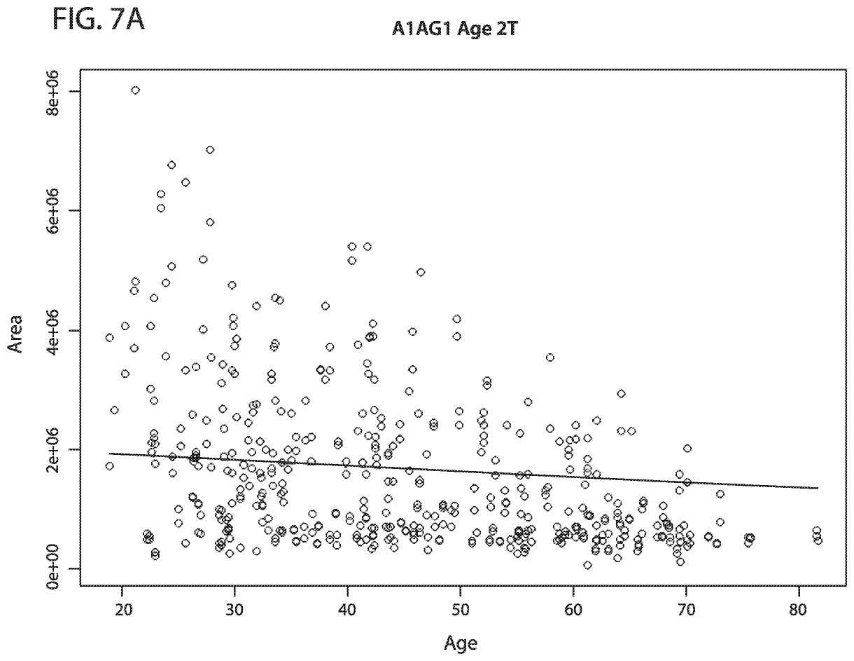

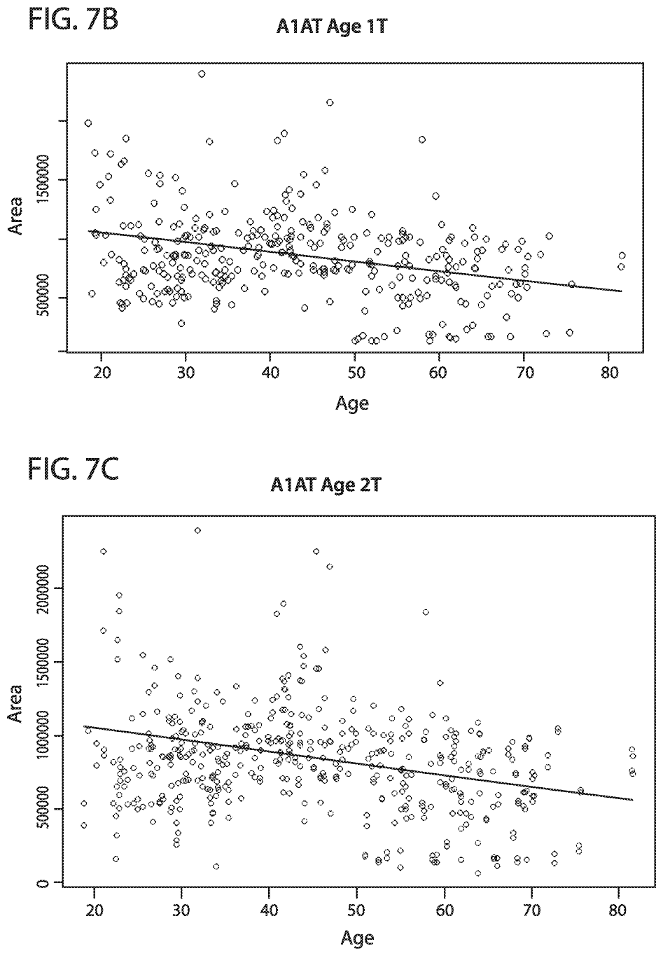

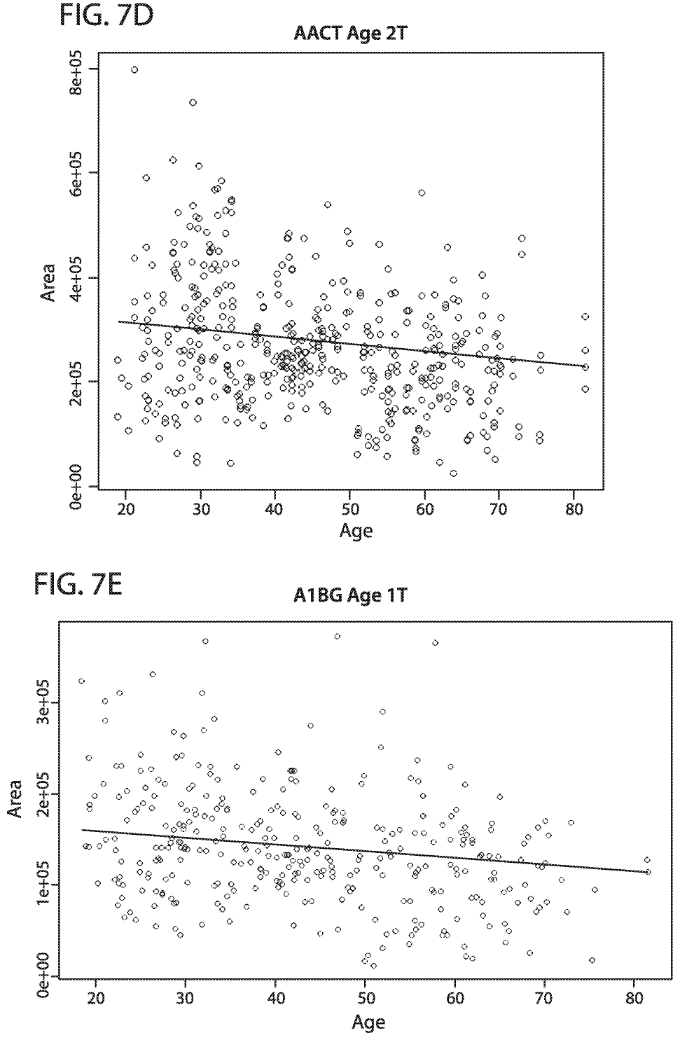

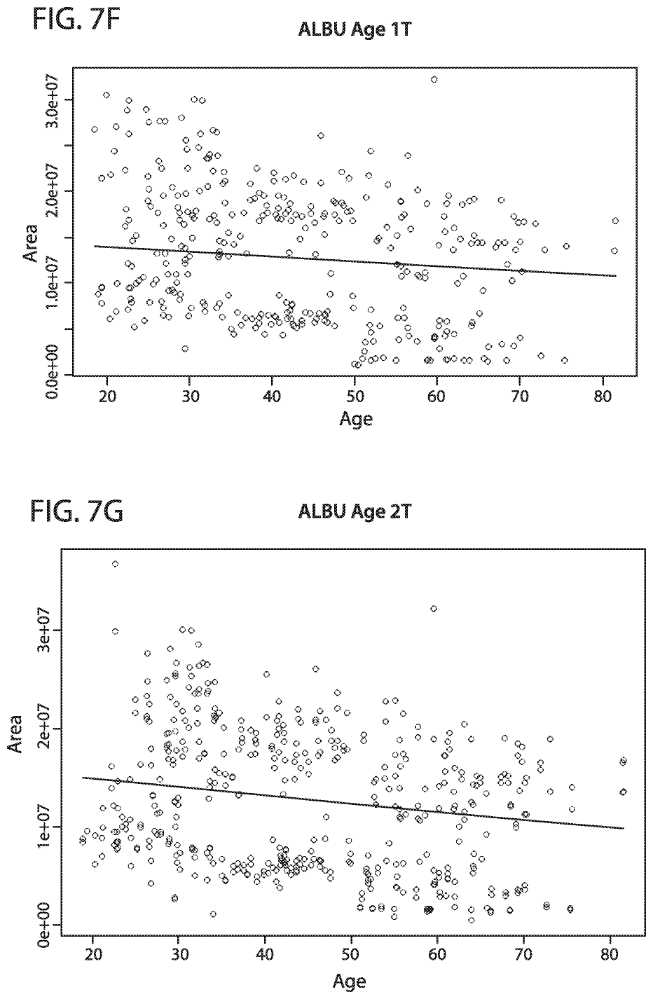

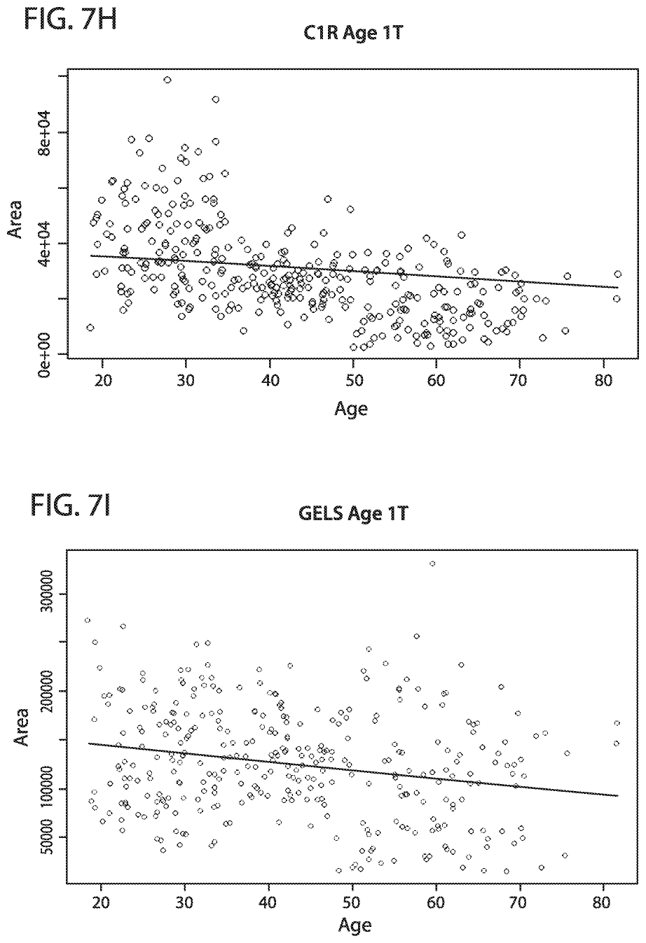

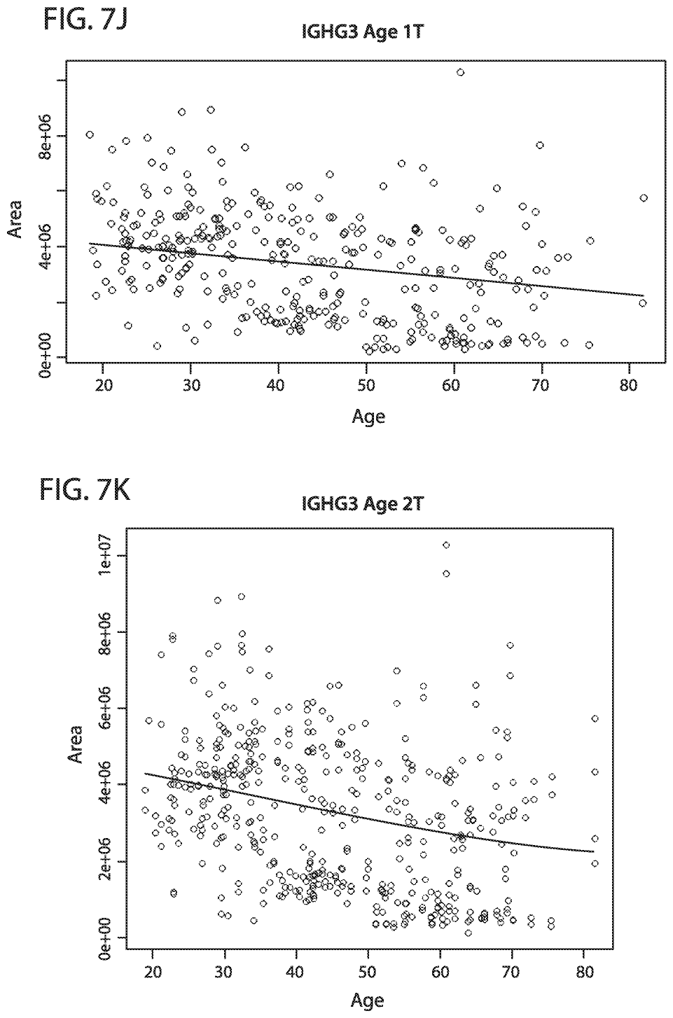

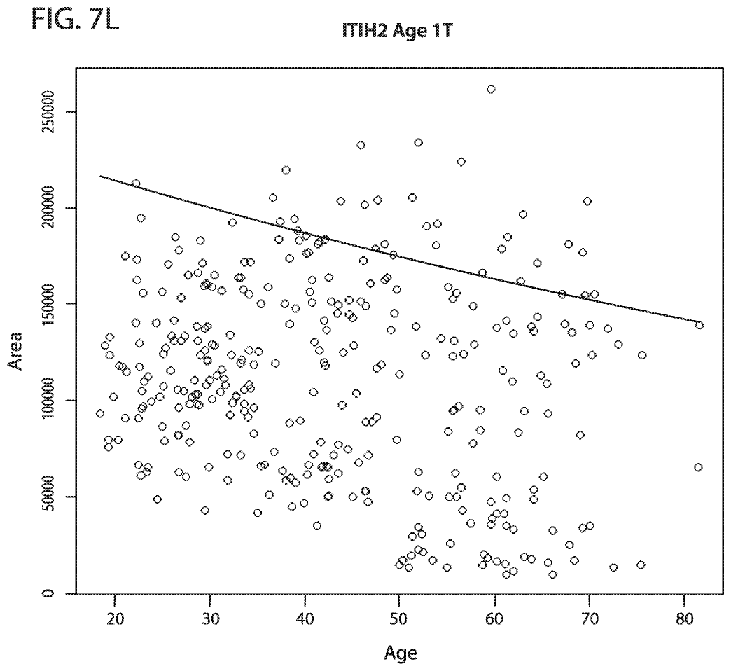

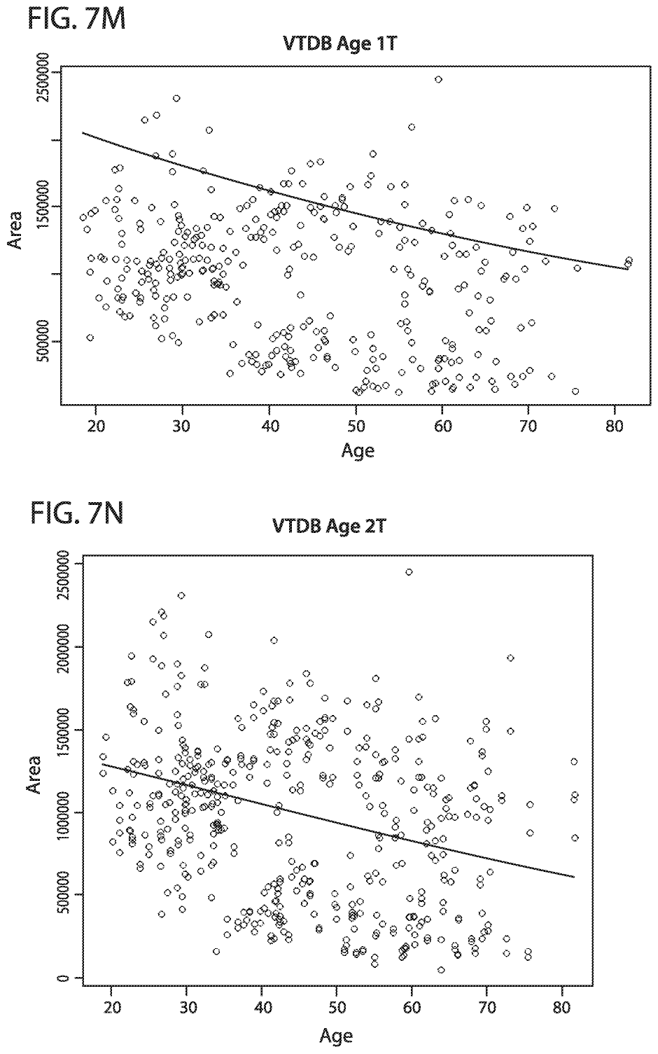

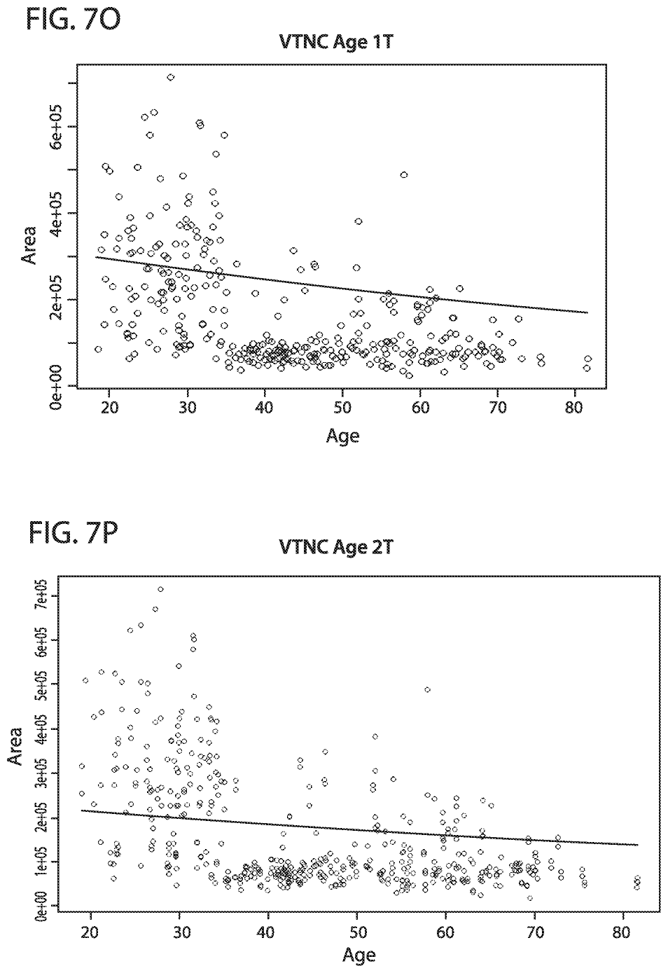

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

For references on mass spectrometry and proteomics, see e.g., Salvatore Sechi, Quantitative Proteomics by Mass Spectrometry (Methods in Molecular Biology) 2^{nd} ed. 2016 Edition, Humana Press (New York, N.Y., 2009); Daniel Martins-de-Souza, Shotgun Proteomics: Methods and Protocols 2014 edition, Humana Press (New York, N.Y., 2014); Jörg Reinders and Albert Sickmann, Proteomics: Methods and Protocols (Methods in Molecular Biology) 2009 edition, Humana Press (New York, N.Y., 2009); and Jörg Reinders, Proteomics in Systems Biology: Methods and Protocols (Methods in Molecular Biology) 1^{st} ed. 2016 edition, Humana Press (New York, N.Y., 2009).

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments, the numbers expressing quantities of ingredients, properties such as concentration, conditions, time, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue; a tissue print; a fingerprint, skin or hair; fragments and derivatives thereof. Non-limiting examples of samples or biological samples include cheek swab; mucus; whole blood, blood, serum; plasma; urine; saliva; semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments the sample is serum. In some embodiments the sample is urine. In some embodiments, the sample is blood, plasma, serum, or urine. In some embodiments, the sample is selected from blood, serum, plasma, urine, and a combination thereof. In some embodiments, the sample is blood, serum, or plasma. In some embodiments, the sample is selected from blood, serum, plasma, and a combination thereof.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device; a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS™) or any collection device where the samples can be stored and delivered (e.g. mailed), and an assay can be performed remotely.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In various embodiments, the subject is mouse or mice. In various embodiments, the subject is human.

In some embodiments, the characteristics of the subject are selected from age, sex, urinary severity index score, Rand Interstitial Cystitis Epidemology (RICE) criteria, Rand Interstitial Cystitis Epidemology (RICE) subtype, and combinations thereof.

In some embodiments, the sex is male. In some embodiments, the sex is female.

In some embodiments, the age is any age. In some embodiments, the age is any age from one day old to 122 years old. In some embodiments, the age is any age from one day old to 100 years old. In some embodiments, the age is any age from 18.5 years old to 81.5 years old.

In some embodiments, the urinary severity index score is any number from 0 to 25. In some embodiments, the urinary severity index score is any number including integers and fractions from 0 to 25. In some embodiments, the urinary severity index score is 0, 1, 2, 3, 4, 5, 6, 7, 8, 9, 10, 11, 12, 13, 14, 15, 16, 17, 18, 19, 20, 21, 22, 23, 24, or 25.

In some embodiments, a subject can be one who has been previously diagnosed with or identified as suffering from or having a syndrome, symptom, disease condition, disease, or disorder in need of treatment or one or more complications related to the syndrome, symptom, disease condition, disease, or disorder. In some embodiments, the subject has previously undergone treatment for the syndrome, symptom, disease condition, disease, disorder, or the one or more complications related to the syndrome, symptom, disease condition, disease, or disorder. In some embodiments, the subject has not previously undergone treatment for the syndrome, symptom, disease condition, disease, disorder, or the one or more complications related to the syndrome, symptom, disease condition, disease, or disorder.

Alternatively, a subject can also be one who has not been previously diagnosed as having a syndrome, symptom, disease condition, disease, or disorder or one or more complications related to the syndrome, symptom, disease condition, disease, or disorder. For example, a subject can be one who exhibits one or more risk factors for a syndrome, symptom, disease condition, disease, or disorder, or one or more complications related to the syndrome, symptom, disease condition, disease, or disorder, or a subject who does not exhibit risk factors.

A “subject in need” of treatment for a particular syndrome, symptom, disease condition, disease, or disorder can be a subject suspected of having that syndrome, symptom, disease condition, disease, or disorder; diagnosed as having that syndrome, symptom, disease condition, disease, or disorder; already treated or being treated for that syndrome, symptom, disease condition, disease, or disorder; not treated for that syndrome, symptom, disease condition, disease, or disorder; or at risk of developing that syndrome, symptom, disease condition, disease, or disorder.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide.” A protein contains at least one polypeptide.

The term “peptide” as used herein refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chern. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn eta/., (1990) Pp. 969-970 in Proc. 11th American Peptide Symposium, ESCOM Science Publishers, The Netherlands, and the like)).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

The term “syndrome” refers to a collection or set of signs or symptoms that characterize or suggest one or more diseases, disease conditions, or disorders. In some embodiments, the syndrome is Urologic Chronic Pelvic Pain Syndrome (UCPPS).

The term “disease” refers to an abnormal condition affecting the body of an organism. For example, the disease or abnormal condition may result from a pathophysiological response to external or internal factors.

The term “disorder” refers to a functional abnormality or disturbance. For example, a disorder may be a disruption of the disease to the normal or regular functions in the body or a part of the body.

The term “disease condition” refers to an abnormal state of health that interferes with the usual activities of feeling of wellbeing.

The term “normal condition” or “healthy condition” refers to a normal state of health.

The term “healthy state” or “normal state” means that the state of a subject (e.g., biological state or health state, etc.) is not abnormal or does not comprise a syndrome, disease, disease condition, or disorder.

A “healthy subject” or “normal subject” is a subject that does not have a syndrome, disease, disease condition, or disorder.

As used herein, the terms “treat”, “treatment”, “treating”, or “amelioration” when used in reference to a syndrome, symptom, disease, disorder or disease condition, refer to both therapeutic treatment and prophylactic or preventative measures, wherein the object is to reverse, alleviate, ameliorate, inhibit, lessen, slow down or stop the progression or severity of a syndrome, symptom, a disease condition, a disease, or a disorder. The term “treating” includes reducing or alleviating at least one adverse effect or symptom of a syndrome, disease condition, a disease, or a disorder. Treatment is generally “effective” if one or more symptoms are reduced. Alternatively, treatment is “effective” if the progression of a syndrome, symptom, disease, disorder or disease condition is reduced or halted. That is, “treatment” includes not just the improvement of symptoms, but also a cessation or at least slowing of progress or worsening of symptoms that would be expected in the absence of treatment. Also, “treatment” may mean to pursue or obtain beneficial results, or lower the chances of the individual developing the syndrome, symptom, disease condition, disease, or disorder even if the treatment is ultimately unsuccessful. Those in need of treatment include those already with the syndrome, symptom, disease condition, disease, or disorder as well as those prone to have the syndrome, symptom, disease condition, disease, or disorder or those in whom the syndrome, symptom, disease condition, disease, or disorder is to be prevented. Treatment also includes a decrease in mortality or an increase in the lifespan of a subject as compared to one not receiving the treatment.

Non-limiting examples of treatments or therapeutic treatments include pharmacological or biological therapies and/or interventional surgical treatments. In some embodiments, the treatment is a treatment for Urologic Chronic Pelvic Pain Syndrome (UCPPS). In some embodiments, the treatment is a treatment for interstitial cystitis/bladder pain syndrome. In some embodiments, the treatment is a treatment for chronic pelvic pain syndrome. In some embodiments, the treatment is a treatment for a symptom of Urologic Chronic Pelvic Pain Syndrome (UCPPS). In some embodiments the treatment is a treatment for a subtype of Urologic Chronic Pelvic Pain Syndrome (UCPPS). In some embodiments, the treatment is a treatment for a symptom of a subtype of Urologic Chronic Pelvic Pain Syndrome (UCPPS).

The term “preventative treatment” means maintaining or improving a healthy state or non-diseased state of a healthy subject or subject that does not have a syndrome, symptom, disease, disease condition, or disorder. The term “preventative treatment” or “health surveillance” also means to prevent or to slow the appearance of symptoms associated with a syndrome, disease condition, disease, or disorder. The term “preventative treatment” also means to prevent or slow a subject from obtaining a syndrome, symptom, disease condition, disease, or disorder.

In various embodiments, the treatment may be formulated for delivery via any route of administration. “Route of administration” may refer to any administration pathway known in the art, including but not limited to aerosol, nasal, via inhalation, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the treatment may be in the form of solutions or suspensions for infusion or for injection, or as lyophilized powders. Via the enteral route, the treatment can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the treatment can be in the form of aerosol, lotion, cream, gel, ointment, suspensions, solutions or emulsions. Methods for these administrations are known to one skilled in the art. In certain embodiments, the treatment is formulated for intravascular, intravenous, or intraarterial administration.

“Beneficial results” or “desired results” may include, but are in no way limited to, lessening or alleviating the severity of the syndrome, symptom, disease, disorder, or disease condition; preventing the syndrome, symptom, disease, disorder, or disease condition from worsening; curing the syndrome, symptom, disease, disorder, or disease condition; preventing the syndrome, symptom, disease, disorder, or disease condition from developing; lowering the chances of a patient developing the syndrome, symptom, disease, disorder, or disease condition; decreasing morbidity and mortality, and prolonging a patient's life or life expectancy. As non-limiting examples, “beneficial results” or “desired results” may be alleviation of one or more symptom(s); diminishment of extent of the deficit; stabilized (i.e., not worsening) state of the syndrome, symptom, disease, disorder, or disease condition; delay or slowing of syndrome, symptom, disease, disorder, or disease condition; and amelioration or palliation of symptoms associated with the syndrome, disease, disorder, or disease condition.

As used herein, the term “administering,” refers to the placement of a treatment as disclosed herein into a subject by a method or route which results in at least partial localization of the treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the treatment may be in the form of solutions or suspensions for infusion or for injection, or as lyophilized powders. Via the enteral route, the treatment can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the treatment can be in the form of aerosol, lotion, cream, gel, ointment, suspensions, solutions or emulsions. In accordance with the present invention, “administering” can be self-administering. For example, it is considered as “administering” that a subject consumes a treatment as disclosed herein.

“Diagnostic” means identifying the presence or nature of a syndrome, symptom, disease condition, disease, or disorder and includes identifying patients who are at risk of developing a syndrome, symptom, disease condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a syndrome, symptom, disease condition, a disease, or a disorder, it suffices if the method provides a positive indication that aids in diagnosis.

By “at risk of” is intended to mean at increased risk of, compared to a reference. Thus a subject carrying a particular marker may have an increased risk for a specific syndrome, symptom, disease condition, disease, or disorder, and be identified as needing further testing. “Increased risk” or “elevated risk” mean any statistically significant increase in the probability, e.g., that the subject has the syndrome, symptom, disease condition, disease, or disorder. The risk is preferably increased by at least 10%, more preferably at least 20%, and even more preferably at least 50% over the reference with which the comparison is being made.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting biomarkers (or an amount of a biomarker), detecting peptides (or an amount of a peptide), detecting proteins (or an amount of a protein), or of detecting a syndrome, detecting a symptom, detecting a disease condition, detecting a disease, or detecting a disorder (e.g. when positive assay results are obtained).

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region. This can include any capture reagents including aptamers.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbant assay), immunoprecipitation, SISCAPA (stable isotope standards and capture by anti-peptide antibodies), Western blot, antibody or aptamer arrays, immuno mass spectrometry assays, etc.

The terms “proteases” and “peptidases” are used interchangeably herein to mean enzymes that breakdown or digest proteins and peptides.

The term “phenotype” as used herein comprises the composite of an organism's observable characteristics or traits, such as its morphology, development, biochemical or physiological properties, phenology, behavior, and products of behavior.

The term “diagnosis,” or “dx,” refers to the identification of the nature and cause of a certain phenomenon. As used herein, a diagnosis typically refers to a medical diagnosis, which is the process of determining which syndrome, disease, disorder or disease condition explains a symptoms and signs. A diagnostic procedure, often a diagnostic test or assay, can be used to provide a diagnosis. A diagnosis can comprise detecting the presence of a syndrome, symptom, disease, disorder, or disease condition.

The term “prognosis,” or “px,” as used herein refers to predicting the likely outcome of a current standing. For example, a prognosis can include the expected duration and course of a syndrome, symptom, disease, disease condition, or disorder, such as progressive decline or expected recovery.

The term “theranosis,” or “tx” as used herein refers to a diagnosis or prognosis used in the context of a medical treatment. For example, theranostics can include diagnostic testing used for selecting appropriate and optimal therapies (or the inverse) based on the context of genetic content or other molecular or cellular analysis. Theranostics includes pharmacogenomics, personalized and precision medicine.

As described herein, the methods of the invention may be used to characterize a phenotype in a sample of interest. The phenotype can be any phenotype of interest that may be characterized using the subject methods. Consider a non-limiting example wherein the phenotype comprises a syndrome, symptom, disease, disease condition, or disorder. In such cases, the characterizing may be providing a diagnosis, prognosis or theranosis for the syndrome, symptom, disease, disease condition, or disorder. In an illustrative embodiment, a sample from a subject is analyzed using the methods of the invention. The analysis is then used to predict or determine the presence, stage, grade, outcome, or likely therapeutic response of a syndrome, symptom, disease, disease condition, or disorder in the subject. The analysis can also be used to assist in making such prediction or determination.

The terms “marker” or “biomarker” are used interchangeably herein, and in the context of the present invention refer to a protein or peptide (for example, protein or peptide associated with a syndrome, symptom, disease, disease condition, or disorder).

In some embodiments, a marker is differentially present or differentially expressed in a sample taken from patients having a specific syndrome, symptom, disease, disease condition or disorder as compared to a reference or reference value or relative to a reference or reference value.

TABLE 1
Proteins with a significant interaction with Sex and RICE subtype.

IC1- 2 T comparison p = 0.0402
APOC1- 1 T comparison p = 0.0057
VTNC- 1 T comparison p = 0.0017
KLKB1- 1 T comparison p = 0.0264,
2 T comparison p = 0.0101

The data in Table 1 shows that the interaction with Sex and RICE Subtype is important for understanding the relationship with protein abundance and RICE Subtype.

The biomarkers of the present invention are the proteins as provided in Table 2 herein.

TABLE 2
Biomarkers (19 proteins)

| | UniProt |
|---|---|
| Protein | Accession No. |
| Complement C1r subcomponent (C1R) | P00736 |
| Alpha-1-antitrypsin (A1AT) | P01009 |
| Alpha-1-antichymotrypsin (AACT) | P01011 |
| Immunoglobulin heavy constant gamma 3 (IGHG3) | P01860 |
| Apolipoprotein A-I (APOA1) | P02647 |
| Apolipoprotein E (APOE) | P02649 |
| Apolipoprotein C-I (APOC1) | P02654 |
| Apolipoprotein C-II (APOC2) | P02655 |
| Alpha-1-acid glycoprotein 1 (A1AG1) | P02763 |
| Serum albumin (ALBU) | P02768 |
| Vitamin D-binding protein (VTDB) | P02774 |
| Plasma kallikrein (KLKB1) | P03952 |
| Vitronectin (VTNC) | P04004 |
| Alpha-1B-glycoprotein (A1BG) | P04217 |
| Plasma protease C1 inhibitor (IC1) | P05155 |
| Gelsolin (GELS) | P06396 |
| Apolipoprotein A-IV (APOA4) | P06727 |
| Complement C4-A (CO4A) | P0C0L4 |
| Inter-alpha-trypsin inhibitor heavy chain H2 (ITIH2) | P19823 |

In some embodiments, biomarkers are detected or measured by an analytical technique. In some embodiments, the analytical technique is mass spectrometry (MS). In some embodiments, the analytical technique is selected from mass spectrometry, immune/aptamer capture-MRM/mass spectrometry, antibody or aptamer arrays, ELISA, immunoprecipitation, SISCPA, Western blot, immunoassay, capture and detection assay, or a combination thereof. In some embodiments, for example, mass spectroscopy may be used to determine multiple biomarkers, and differences between individual biomarkers and/or the partial or complete profile may be used for diagnosis.

In some embodiments, biomarkers are detected or measured by mass spectrometry, immune/aptamer capture-MRM/mass spectrometry, antibody or aptamer arrays, ELISA, immunoprecipitation, SISCPA, Western blot, immunoassay, capture and detection assay, or a combination thereof. In some embodiments, amounts of biomarkers or levels of biomarkers are detected or measured by mass spectrometry, immune/aptamer capture-MRM/mass spectrometry, antibody or aptamer arrays, ELISA, immunoprecipitation, SISCPA, Western blot, immunoassay, capture and detection assay, or a combination thereof.

In some embodiments, biomarkers are detected or measured by mass spectrometry, an immunoassay, or a capture and detection assay. In some embodiments, amounts of biomarkers or levels of biomarkers are detected or measured by mass spectrometry, an immunoassay, or a capture and detection assay.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of a particular syndrome, symptom, disease, disease condition, or disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the syndrome, symptom, disease, disease condition, or disorder sought to be diagnosed. A control amount can be either in absolute amount (e.g., μg/ml) or a relative amount (e.g., relative intensity of signals).

The term “differentially present” or “change in level” or “change in amount” or “differentially expressed” refers to differences in the quantity, amount, level and/or the frequency of a marker present or expressed in a sample taken from subjects having a specific syndrome, symptom, disease, disease condition, or disorder as compared to a reference.

For example, in some embodiments a marker can be detected, measured, present, or expressed in samples from subjects with the syndrome, symptom, disease, disease condition, or disorder compared to a reference.

For example, in some embodiments a marker can be detected, measured, present, or expressed at an elevated level or amount (or increased level or amount) in samples from subjects with the syndrome, symptom, disease, disease condition, or disorder compared to a reference or relative to a reference.

In some embodiments, a marker can be detected, measured, present or expressed at a decreased level or amount in samples from subjects with the syndrome, symptom, disease, disease condition, or disorder compared to a reference or relative to a reference.

In some embodiments, a marker can be detected, measured, present, or expressed at a higher frequency in samples from subjects with the syndrome, symptom, disease, disease condition, or disorder compared to a reference or relative to a reference.

In some embodiments, a marker can be detected, measured, present, or expressed at a lower frequency in samples from subjects with the syndrome, symptom, disease, disease condition, or disorder compared to a reference or relative to a reference.

In some embodiments, a marker can be differentially present or differentially expressed in terms of quantity, amount, level, frequency, or a combination thereof. In some embodiments, a marker can be differentially present or differentially expressed as a ratio of differences between two or more specific markers.

In some embodiments, a marker is differentially present or differentially expressed in a sample if the amount of the marker in the sample is statistically significantly different from the amount of the marker in another sample (e.g., a reference or reference sample). For example, a marker is differentially present or differentially expressed if it is present or expressed at least about 120%, at least about 130%, at least about 150%, at least about 180%, at least about 200%, at least about 300%, at least about 500%, at least about 700%, at least about 900%, or at least about 1000% greater or less than it is present or expressed in the other sample (e.g., a reference or reference sample), or if it is detectable in one sample and not detectable in the other.

Alternatively, or additionally, a marker is differentially present or differentially expressed between samples if the frequency of detecting the marker in samples of subjects suffering from a particular syndrome, symptom, disease, disease condition, or disorder, is statistically significantly higher or lower than in the reference or reference sample. For example, a biomarker is differentially present or differentially expressed between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples than the other set of samples. These exemplary values notwithstanding, it is expected that a skilled practitioner can determine cut-off points, etc. that represent a statistically significant difference to determine whether the marker is differentially present or differentially expressed.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides.

By “binding assay” is meant a biochemical assay wherein the biomarkers are detected by binding to an agent, such as an antibody, through which the detection process is carried out. The detection process may involve radioactive or fluorescent labels, and the like. The assay may involve immobilization of the biomarker, or may take place in solution.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

In some embodiments, the reference is a baseline value for the amount of the biomarker, wherein the baseline value is from at least one sample obtained from the subject at an earlier point in time.

In some embodiments, the reference is a reference sample from a control subject, wherein the control subject does not have Urologic Chronic Pelvic Pain Syndrome (UCPPS).

In some embodiments, the reference is a reference sample from a control subject, wherein the control subject has Urologic Chronic Pelvic Pain Syndrome (UCPPS).

In some embodiments, the reference is a range of reference values for the amount of the biomarker, wherein the range of reference values is from at least one reference sample obtained from at least one healthy subject.

In some embodiments, the reference is a range of reference values for the amount of the biomarker, wherein the range of reference values is from at least one sample obtained from the subject at an earlier point in time.

In some embodiments, the reference is a range of reference values for the amount of the biomarker, wherein the range of reference values is from at least one reference sample obtained from at least one reference subject, wherein the reference subject has Urologic Chronic Pelvic Pain Syndrome (UCPPS).

In some embodiments, the subject has a syndrome, symptom, disease, disease condition, disorder, or a combination thereof. In some embodiments, the syndrome is Urologic Chronic Pelvic Pain Syndrome (UCPPS). In some embodiments, the symptom is selected from (a) painful filling, (b) painful urgency, (c) both painful filling and painful urgency, and (d) neither painful filling nor painful urgency.

In some embodiments, a symptom of the Rand Interstitial Cystitis Epidemology (RICE) subtype is selected from (a) painful filling, (b) painful urgency, (c) both painful filling and painful urgency, and (d) neither painful filling nor painful urgency.

In some embodiments, a subtype of the Rand Interstitial Cystitis Epidemology (RICE) subtype is selected from (a) painful filling, (b) painful urgency, (c) both painful filling and painful urgency, and (d) neither painful filling nor painful urgency.

In some embodiments, the Rand Interstitial Cystitis Epidemology (RICE) subtypes are selected from (a) painful filling, (b) painful urgency, (c) both painful filling and painful urgency, and (d) neither painful filling nor painful urgency.

In some embodiments, the Rand Interstitial Cystitis Epidemology (RICE) criteria are selected from (a) painful filling, (b) painful urgency, (c) both painful filling and painful urgency, and (d) neither painful filling nor painful urgency.

In some embodiments, the symptom is a symptom of a syndrome. In some embodiments, the symptom is a symptom of a subtype of a syndrome. In some embodiments, the symptom is a symptom of a disease. In some embodiments, the symptom is a symptom of a disorder. In some embodiments, the symptom is a symptom of a disease condition. In some embodiments, the symptom is a subtype of a syndrome.

In some embodiments, the subtype is a subtype of a syndrome. In some embodiments, the subtype is a subtype of a disease. In some embodiments, the subtype is a subtype of a disorder. In some embodiments, the subtype is a subtype of a disease condition.

In some embodiments, the subtype is a subtype of a syndrome subtype. In some embodiments, the subtype is a subtype of a disease subtype. In some embodiments, the subtype is a subtype of a disorder subtype. In some embodiments, the subtype is a subtype of a disease condition subtype.

In some embodiments, the subject is a test subject, and the test subject has a syndrome, symptom, disease, disease condition, disorder, or a combination thereof.

The terms “painful filling”, “painful urgency”, “flare”, “flare status”, “urinary severity”, “Urinary Severity Index”, “urinary severity index score”, “interstitial cystitis (IC) subtype”, “Rand Interstitial Cystitis Epidemology (RICE) criteria”, “Rand Interstitial Cystitis Epidemology (RICE) subtype”, and “pelvic pain and beyond” as defined below are based on work by the Multidisciplinary Approach to the Study of Chronic Pelvic Pain (+MAPP) network. The MAPP network identified specific subtypes of patients with urogenital chronic pelvic pain syndrome (UCPPS).

The term “painful filling” refers to a patient that experiences pain specifically as their bladder begins to fill.

The term “painful urgency” refers to patients that have the urge to urinate that is accompanied by bladder pain. They are driven to void by pain.

The term “flare” refers to painful exacerbation of baseline bladder pain symptoms. Can last from minutes to days to weeks.

The term “flare status” refers to a binary variable (they are either in a flare or not in a symptom flare).

The term “urinary severity” refers to the severity of a patient's urinary symptoms, specifically painful filling and painful urgency. This is considered separate from patients who have bladder pain that is not associated with filling or urgency.

The term “Urinary Severity Index” refers to a validated measure of urinary severity, as defined above.

The term “urinary severity index score” refers to the score/measurement system used for urinary severity index.

The term “interstitial cystitis (IC) subtype” refers to one of many types of IC. These include those with painful filling and urgency, pelvic pain only, pelvic pain and beyond, and whether they have flares.

The term “Rand Interstitial Cystitis Epidemology (RICE) criteria” specifically refers to painful filling and painful urgency, as defined originally by RICE study.

The term “Rand Interstitial Cystitis Epidemology (RICE) subtype” refers to a subtype of Urologic Chronic Pelvic Pain Syndrome (UCPPS) as defined by RICE study.

The term “pelvic pain and beyond” refers to a subtype of Urologic Chronic Pelvic Pain Syndrome (UCPPS) in which patients have pelvic pain as well as pain in other body parts, such as migraine, back pain, etc.

Urologic chronic pelvic pain syndrome (UCPPS) encompasses both interstitial cystitis/bladder pain syndrome and chronic prostatitis/chronic pelvic pain syndrome. A lack of understanding of the molecular mechanisms underlying UCPPS has been a challenge and dilemma for diagnosis and treatment leading to a delay in basic and translational research focused on biomarker and drug discovery, clinical therapy, and preventive strategies. Without being bound by theory, we hypothesized that UCPPS is associated with specific protein expression patterns in the blood.

Mass Spectrometry (MS)

As used herein, SRM stands for selected reaction monitoring. As used herein, MRM stands for multiple reaction monitoring. As used herein, PRM stands for parallel reaction monitoring. As used herein, SWATH stands for sequential window acquisition of all theoretical fragment ion spectra. As used herein, DIA stands for data-independent analysis. As used herein, MS stands for mass spectrometry. As used herein, SIL stands for stable isotope-labeled.

As used herein, “MS data” can be raw MS data obtained from a mass spectrometer and/or processed MS data in which peptides and their fragments (e.g., transitions and MS peaks) are already identified, analyzed and/or quantified. MS data can be Selective Reaction Monitoring (SRM) data, Multiple Reaction Monitoring (MRM) data, Parallel Reaction Monitoring (PRM) data, Shotgun CID MS data, Original DIA MS Data, MSE MS data, p2CID MS Data, PAcIFIC MS Data, AIF MS Data, XDLA MS Data, SWATH MS data, or FT-ARM MS Data, Data Dependent Acquisition (DDA) data, Data Independent Acquisition (DIA) data or their combinations.

In some embodiments, PRM uses a quadrupole time-of-flight (QTOF) or hybrid quadrupole-orbitrap (QOrbitrap) mass spectrometer to carry out the peptides/proteins quantitation. Examples of quadrupole time-of-flight (QTOF) include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QTof Quadrupole Time-of-Flight Mass Spectrometry (Waters). Examples of hybrid quadrupole-orbitrap (QObitrap) include but are not limited to: Q Exactive™ Hybrid Quadrupole-Orbitrap Mass Spectrometer (the Thermo Scientific); or Orbitrap Fusion™ Tribrid™ (the Thermo Scientific).

SWATH MS is a data independent acquisition (DIA) method which aims to complement traditional mass spectrometry-based proteomics techniques such as shotgun and SRM methods. In essence, it allows a complete and permanent recording of all fragment ions of the detectable peptide precursors present in a biological sample. It thus combines the advantages of shotgun (high throughput) with those of SRM (high reproducibility and consistency).

In some embodiments, the developed methods herein can be applied to the quantification of polypeptides(s) or protein(s) in a sample (e.g., biological sample). Any kind of biological samples comprising polypeptides or proteins can be the starting point and be analyzed by the methods herein. Indeed, any protein, polypeptide, or peptide containing sample can be used for and analyzed by the methods produced here. The methods herein can also be used with peptide mixtures obtained by digestion. Digestion of a polypeptide or protein includes any kind of cleavage strategies, such as, enzymatic, chemical, physical or combinations thereof.

The deciding factors of which polypeptide or protein will be the one of interest varies. It can be decided by performing a literature search and identifying proteins that are functionally related, are candidate protein biomarkers which can be used in screening for drug discovery, biomarker discovery and/or disease clinical phase trials or are diagnostic markers to screen for pharmaceutical/medical purposes. The polypeptide or protein of interest may be determined by experimental analysis.

According to some embodiments, the following parameters of the methods provided herein are determined: trypsin (or other protease) digestion and peptide clean up, best responding polypeptides, best responding proteins, best responding peptides, best responding fragments, fragment intensity ratios (increased high and reproducible peak intensities), optimal collision energies, and all the optimal parameters to maximize sensitivity and/or specificity of the methods.

In other embodiments, quantification of the polypeptides and/or of the corresponding proteins or activity/regulation of the corresponding proteins is desired. A selected peptide is labeled with a stable-isotope and used as an internal standard to achieve absolute quantification of a protein of interest. The addition of a quantified stable-labeled peptide analogue of the tag to the peptide sample in known amount; and subsequently the tag and the peptide of interest is quantified by mass spectrometry and absolute quantification of the endogenous levels of the proteins is obtained.

The present invention can be beneficial for the pharmaceutical industry (e.g. drug development and assessment), the biotechnology industry (e.g. assay design and development and quality control), and in clinical applications (e.g. identification of biomarkers of disease and quantitative analysis for diagnostic, prognostic and/or therapeutic use).

In some embodiments, the mass spectrometer is a triple quadrupole mass spectrometer. In some embodiments the mass spectrometer is a Triple-Time Of Flight (Triple-TOF) mass spectrometer configured for SWATH or a Q-Exactive mass spectrometer (Thermo Scientific), or any instrument with sufficiently high scan speed and a quadrupole mass filter to perform data independent acquisition. Examples of triple quadrupole mass spectrometers (TQMS) that can perform MRM/SRM/SIM include but are not limited to: QTRAP® 6500 and 5500 System (Sciex); Triple QTriple Quad 6500 System (Sciex); Agilent 6400 Series Triple Quadrupole LC/MS systems; Thermo Scientific™ TSQ™ Triple Quadrupole system; quadrupole time-of-flight (QTOF) mass spectrometers, or hybrid quadrupole-orbitrap (QOrbitrap) mass spectrometers to carry out the peptides/proteins quantitation. Examples of quadrupole time-of-flight (QTOF) mass spectrometers include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QTof Quadrupole Time-of-Flight Mass Spectrometry (Waters). Examples of hybrid quadrupole-orbitrap (QObitrap) mass spectrometers include but are not limited to: Q Exactive™ Hybrid Quadrupole-Orbitrap Mass Spectrometer (the Thermo Scientific); or Orbitrap Fusion™ Tribrid™ (the Thermo Scientific).

In some embodiments, the mass spectrometry is tandem mass spectrometry (MS/MS). In some embodiments, the mass spectrometry is liquid chromatography-tandem mass spectrometry (LC-MS/MS). In some embodiments, the mass spectrometry is liquid chromatography-selected reaction monitoring-mass spectrometry (LC-SRM-MS). In some embodiments, the mass spectrometry is liquid chromatography-multiple reaction monitoring-mass spectrometry (LC-MRM-MS). In some embodiments, the mass spectrometry is selected reaction monitoring mass spectrometry. In some embodiments, the mass spectrometry is multiple reaction monitoring (MRM) mass spectrometry. In some embodiments, the mass spectrometry is parallel reaction monitoring (PRM) mass spectrometry.

In various embodiments, the peptides are derived by proteolysis or chemical cleavage of the polypeptide or protein. In an embodiment, a protease is utilized to cleave the polypeptide or protein into peptides. For example, the protease is trypsin. In additional embodiments, proteases or cleavage agents may be used including but not limited to trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof.

In some embodiments, the proteins or the amounts or levels of the proteins can be measured and/or detected as intact proteins by mass spectrometry. In some embodiments, the proteins or the amounts or levels of the proteins can be measured and/or detected as intact proteins by mass spectrometry using a mass spectrometer. In some embodiments, no digestion of the proteins is required prior to mass spectrometry.

In some embodiments, the proteins or the amounts or levels of the proteins can be measured and/or detected as intact proteins using an immunoassay, or a capture and detection assay. In some embodiments, no digestion of the proteins is required prior to performing an immunoassay, or capture and detection assay.

In some embodiments, the proteins or the amounts or levels of the proteins can be measured and/or detected as intact proteins by mass spectrometry using a mass spectrometer, immunoassay, capture and detection assay, or combination thereof. In some embodiments, no digestion of the proteins is required prior to mass spectrometry, immunoassay, capture and detection assay or combination thereof.

In some embodiments, the proteins are digested before the proteins or amounts or levels of the proteins are measured and/or detected by mass spectrometry, immunoassay, capture and detection assay, or combination thereof.

In some embodiments, peptides and/or amounts or levels of peptides and/or protein fragments and/or amounts or levels of protein fragments can be measured and/or detected by mass spectrometry, immunoassay, capture and detection assay, or combination thereof.

In an embodiment, the measurement and/or detection of a peptide is improved by changing the conditions for fragmenting that peptide prior to detecting a multiplicity of the peptides with the mass spectrometer. In exemplary embodiments, the fragmentation condition is the collision energy.

In various other embodiments, the method further comprises adding a stable isotope-labeled peptide to the sample prior to mass spectrometry. In some embodiments, the absolute amount of a peptide in the sample is determined by comparing the MS signals of natural and stable isotope-labeled peptides. In various other embodiments, the method further comprises adding a labeled peptide to the sample prior to mass spectrometry. In some embodiments, the absolute amount of a peptide in the sample is determined by comparing the MS signals of natural and labeled peptides.

In various other embodiments, the method further comprises adding a stable isotope-labeled protein to the sample prior to mass spectrometry. In some embodiments, the absolute amount of a protein in the sample is determined by comparing the MS signals of natural and stable isotope-labeled proteins.

In some embodiments, the amount or level of the protein (biomarker) present in a sample is determined by detecting an amount of a peptide in the sample, and correlating the amount of the peptide detected in the sample to the amount of the protein (biomarker) present in the sample. In some embodiments, the amount or level of the protein (biomarker) present in a reference is determined by detecting an amount of a peptide in the reference, and correlating the amount of the peptide detected in the reference to the amount of the protein (biomarker) present in the reference.

In some embodiments, the amount or level of the protein (biomarker) present in a sample is determined by detecting an amount of a protein fragment in the sample, and correlating the amount of the protein fragment detected in the sample to the amount of the protein (biomarker) present in the sample. In some embodiments, the amount or level of the protein (biomarker) present in a reference is determined by detecting an amount of a protein fragment in the reference, and correlating the amount of the protein fragment detected in the reference to the amount of the protein (biomarker) present in the reference.

In various other embodiments, transitions for each protein or peptide or protein fragment or peptide fragment with high and reproducible peak intensities are identified. In other embodiments, the collision energy for each transition is optimized. In other embodiments, mass spectrometry comprises selected reaction monitoring (SRM), or multiple reaction monitoring (MRM). In other embodiments, SRM or MRM is performed on a triple quadrapole mass spectrometer. In other embodiments, the peptides or protein fragments uniquely associated with the polypeptide or protein of interest are those with high correlations, strong signals, high signal/noise and/or sequences unique to the protein of interest.

Selected-ion monitoring (SIM) or selected reaction monitoring (SRM) or multiple reaction monitoring (MRM) provide the simplest method set up and the most selective and sensitive quantification. SRM/MRM/SIM is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to: QTRAP® 6500 and 5500 System (Sciex); Triple QTriple Quad 6500 System (Sciex); Agilent 6400 Series Triple Quadrupole LC/MS systems; or Thermo Scientific™ TSQ™ Triple Quadrupole system.

In various other embodiments, stable isotope-labeled peptide standards for absolute quantification are used. In other embodiments, the peptide labeled with a stable isotope is used as an internal standard to obtain absolute quantification of the polypeptide or protein of interest. In other embodiments, the peptides are quantified and then the amount of the parent protein present is inferred before digesting the sample with trypsin. In other embodiments, MS responses are used to determine an upper limit of quantification (ULOQ) and a lower limit of quantification (LLOQ).

In various embodiments, acquiring MS data comprises operating a TripleTOF mass spectrometer, a triple quadrupole mass spectrometer, a liquid chromatography-mass spectrometry (LC-MS) system, a gas chromatography-mass spectrometry (GC-MS) system, or a tandem mass spectrometry (MS/MS) system, a dual time-of-flight (TOF-TOF) mass spectrometer, or a combination thereof.

In various embodiments, mass spectrometry comprises operating a mass spectrometer. In various embodiments, acquiring MS data comprises operating a mass spectrometer. Examples of the mass spectrometer include but are not limited to high-resolution instruments such as Triple-TOF, Orbitrap, Fourier transform, and tandem time-of-flight (TOF/TOF) mass spectrometers; and high-sensitivity instruments such as triple quadrupole, ion trap, quadrupole TOF (QTOF), and Q trap mass spectrometers; and their hybrid and/or combination. High-resolution instruments are used to maximize the detection of peptides with minute mass-to-charge ratio (m/z) differences. Conversely, because targeted proteomics emphasize sensitivity and throughput, high-sensitivity instruments are used. In some embodiments, the mass spectrometer is a TripleTOF mass spectrometer. In some embodiments, the mass spectrometer is a triple quadrupole mass spectrometer.

In various embodiments, the MS data is collected by a targeted acquisition method. Examples of the targeted acquisition method include but are not limited to Selective Reaction Monitoring (SRM) and/or Multiple Reaction Monitoring (MRM) methods. In various embodiments, acquiring MS data comprises acquiring Selective Reaction Monitoring (SRM) data and/or Multiple Reaction Monitoring (MRM) data.

In various embodiments, the MS data is collected by a data independent acquisition (DIA) method. In various embodiments, the MS data is collected by data dependent acquisition (DDA) method.

Additional non-limiting examples of mass spectrometry include collision-induced dissociation (CID), higher-energy collisional dissociation (HCD), electron-transfer dissociation (ETD), etc.

Non-Limiting Embodiments of the Invention

In various embodiments, the present invention provides a method for determining status of Urologic Chronic Pelvic Pain Syndrome (UCPPS) in a subject, comprising: obtaining a sample from the subject, wherein the subject has Urologic Chronic Pelvic Pain Syndrome (UCPPS), and wherein the sample is selected from blood, serum, plasma, urine, and a combination thereof; detecting an amount of at least one biomarker in the sample, wherein the biomarker is a protein selected from A1AT, APOC2, APOA4, ITIH2, VTNC, APOC1, CO4A, GEL, IC1, AACT, APOA1, A1BG, APOE, C1R, IGHG3, A1AG1, ALBU, VTDB, KLKB1, and a combination thereof; and comparing the amount of the at least one biomarker in the sample from the subject to an amount of the at least one biomarker in a reference to determine the status of Urologic Chronic Pelvic Pain Syndrome (UCPPS) in a subject.

In some embodiments, the reference is selected from: (i) a baseline value for the amount of the biomarker, wherein the baseline value is from at least one sample obtained from the subject at an earlier point in time; (ii) a reference sample from a control subject, wherein the control subject does not have Urologic Chronic Pelvic Pain Syndrome (UCPPS); (iii) a reference sample from a control subject, wherein the control subject has Urologic Chronic Pelvic Pain Syndrome (UCPPS); (iv) a range of reference values for the amount of the biomarker, wherein the range of reference values is from at least one reference sample obtained from at least one healthy subject; (v) a range of reference values for the amount of the biomarker, wherein the range of reference values is from at least one sample obtained from the subject at an earlier point in time; and (vi) a range of reference values for the amount of the biomarker, wherein the range of reference values is from at least one reference sample obtained from at least one reference subject, wherein the reference subject has Urologic Chronic Pelvic Pain Syndrome (UCPPS).

In some embodiments, an increase in the amount of the at least one biomarker in the sample from the subject relative to the amount of the at least one biomarker in the reference is used to determine a Rand Interstitial Cystitis Epidemiology (RICE) subtype in the subject, wherein the biomarker is selected from A1AT, ITIH2, VTNC, APOC1, IC1, AACT, APOA1, A1BG, APOE, C1R, IGHG3, A1AG1, ALBU, VTDB, and KLKB1.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the biomarker is selected from A1AT, APOC2, APOA4, ITIH2, and VTNC, and wherein severity of urinary symptoms of Urologic Chronic Pelvic Pain Syndrome (UCPPS) in the subject is scored according to the urinary severity index.

In some embodiments, the baseline value for the amount of the biomarker in the sample from the subject is dependent on a characteristic of the subject, wherein the biomarker is selected from A1AT, ITIH2, and VTNC, and wherein the characteristic of the subject is selected from age, Rand Interstitial Cystitis Epidemiology (RICE) subtype, urinary severity index score, and a combination thereof.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the baseline value for the amount of the biomarker in the sample from the subject is dependent on a characteristic of the subject, wherein the biomarker is selected from A1AT, ITIH2, VTNC, AACT, A1BG, IGHG3, A1AG1, ALBU, and VTDB, and wherein the characteristic of the subject is selected from age, sex, and a combination thereof.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the method further comprises using the amount of the biomarker in the sample from the subject to distinguish between (a) a diagnosis of pelvic pain in the subject, and (b) a diagnosis of pelvic pain and beyond in the subject, wherein the biomarker is selected from APOC1 and CO4A.

In some embodiments, the biomarker is APOC1, and wherein the amount of the biomarker in the sample from the subject is dependent on a characteristic of the subject, wherein the characteristic of the subject is selected from sex, Rand Interstitial Cystitis Epidemiology (RICE) subtype, and a combination thereof.

In some embodiments, a symptom of the Rand Interstitial Cystitis Epidemiology (RICE) subtype is selected from (a) painful filling, (b) painful urgency, (c) both painful filling and painful urgency, and (d) neither painful filling nor painful urgency.

In some embodiments, the method further comprises using the amount of the biomarker in the sample from the subject to assist in diagnosing flare status of Urologic Chronic Pelvic Pain Syndrome (UCPPS) in the subject, wherein the biomarker is GELS.

In some embodiments, the amount of the biomarker in the sample from the subject is increased relative to the amount of the biomarker in the reference, wherein the biomarker is selected from C1R and A1AT, and wherein the reference is a reference sample from a control subject, wherein the control subject does not have Urologic Chronic Pelvic Pain Syndrome (UCPPS).

In some embodiments, the biomarker is A1AT, and wherein the amount of the biomarker in the sample from the subject is dependent on a characteristic of the subject, wherein the characteristic of the subject is selected from age, sex, urinary severity index score, and a combination thereof.

In some embodiments, the biomarker is C1R, and wherein the amount of the biomarker in the sample from the subject is dependent on a characteristic of the subject, wherein the characteristic of the subject is selected from age, sex, and a combination thereof.

In some embodiments, the biomarker is selected from APOA1, A1BG, APOE, A1AT, IGHG3, ALBU, VTDB, VTNC, and ITIH2, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) both painful filling and painful urgency, and (b) neither painful filling nor painful urgency.

In some embodiments, the biomarker is selected from AACT, A1BG, C1R, A1AT, IGHG3, VTDB, VTNC, IC1, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) painful urgency, and (b) neither painful filling nor painful urgency.

In some embodiments, the biomarker is A1AT, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) painful urgency, and (b) both painful filling and painful urgency.

In some embodiments, the biomarker is selected from A1AT, A1AG1, and VTNC, and wherein the amount of the biomarker in the sample from the subject relative to the amount of the biomarker in the reference determines the subject's Rand Interstitial Cystitis Epidemiology (RICE) subtype.

In some embodiments, a symptom of the Rand Interstitial Cystitis Epidemiology (RICE) subtype is selected from (a) painful filling, and (b) painful urgency.

In some embodiments, the amount of the biomarker in the sample from the subject is dependent on a characteristic of the subject, wherein the characteristic of the subject is sex, and wherein the biomarker is selected from IC1, AACT, APOC1, and KLKB1.

In some embodiments, the amount of the biomarker in the sample from the subject is dependent on a characteristic of the subject, wherein the characteristic of the subject is age, and wherein the biomarker is selected from A1AG1 and ALBU.

In some embodiments, the amount of the biomarker in the sample from the subject is dependent on a characteristic of the subject, wherein the characteristic of the subject is selected from sex, age, and a combination thereof, and wherein the biomarker is selected from A1BG, IGHG3, and VTDB.

In some embodiments, the amount of the biomarker in the sample from the subject is dependent on a characteristic of the subject, wherein the characteristic of the subject is selected from age, urinary severity index score, and a combination thereof, and wherein the biomarker is ITIH2.

In some embodiments, the amount of the biomarker in the sample from the subject is dependent on a characteristic of the subject, wherein the characteristic of the subject is selected from age, sex, urinary severity index score, and a combination thereof, and wherein the biomarker is selected from A1AT and VTNC.

In some embodiments, the method further comprises administering a treatment for Urologic Chronic Pelvic Pain Syndrome (UCPPS) to the subject.

In some embodiments, the method further comprises administering a treatment for a symptom of Urologic Chronic Pelvic Pain Syndrome (UCPPS) to the subject.

In some embodiments, the amount of the biomarker is detected by mass spectrometry, an immunoassay, or a capture and detection assay.

In some embodiments, the mass spectrometry comprises operating a mass spectrometer.

In some embodiments, the immunoassay comprises performing the immunoassay.

In some embodiments, the capture and detection assay comprises performing the capture and detection assay.

In some embodiments, the biomarker is an isoform, single-nucleotide polymorphism, or fragment of the protein. In some embodiments, the biomarker is an isoform, single-nucleotide polymorphism, peptide, or fragment of the protein

In some embodiments, the protein comprises a post-translational modification. In some embodiments, the protein fragment comprises a post-translational modification. In some embodiments, the peptide comprises a post-translational modification.

In some embodiments, the subject is human.

Some embodiments of the present invention can be defined as any of the following numbered paragraphs:

1. A method for determining status of Urologic Chronic Pelvic Pain Syndrome (UCPPS) in a subject, comprising: obtaining a sample from the subject, wherein the subject has Urologic Chronic Pelvic Pain Syndrome (UCPPS), and wherein the sample is selected from blood, serum, plasma, urine, and a combination thereof; detecting an amount of at least one biomarker in the sample, wherein the biomarker is a protein selected from A1AT, APOC2, APOA4, ITIH2, VTNC, APOC1, CO4A, GEL, IC1, AACT, APOA1, A1BG, APOE, C1R, IGHG3, A1AG1, ALBU, VTDB, KLKB1, and a combination thereof; and comparing the amount of the at least one biomarker in the sample from the subject to an amount of the at least one biomarker in a reference to determine the status of Urologic Chronic Pelvic Pain Syndrome (UCPPS) in a subject.
2. The method of paragraph 1, wherein the reference is selected from: (i) a baseline value for the amount of the biomarker, wherein the baseline value is from at least one sample obtained from the subject at an earlier point in time; (ii) a reference sample from a control subject, wherein the control subject does not have Urologic Chronic Pelvic Pain Syndrome (UCPPS); (iii) a reference sample from a control subject, wherein the control subject has Urologic Chronic Pelvic Pain Syndrome (UCPPS); (iv) a range of reference values for the amount of the biomarker, wherein the range of reference values is from at least one reference sample obtained from at least one healthy subject; (v) a range of reference values for the amount of the biomarker, wherein the range of reference values is from at least one sample obtained from the subject at an earlier point in time; and (vi) a range of reference values for the amount of the biomarker, wherein the range of reference values is from at least one reference sample obtained from at least one reference subject, wherein the reference subject has Urologic Chronic Pelvic Pain Syndrome (UCPPS).
3. The method of paragraph 2, wherein an increase in the amount of the at least one biomarker in the sample from the subject relative to the amount of the at least one biomarker in the reference is used to determine a Rand Interstitial Cystitis Epidemiology (RICE) subtype in the subject, wherein the biomarker is selected from A1AT, ITIH2, VTNC, APOC1, IC1, AACT, APOA1, A1BG, APOE, C1R, IGHG3, A1AG1, ALBU, VTDB, and KLKB1.
4. The method of paragraph 3, wherein a symptom of the Rand Interstitial Cystitis Epidemiology (RICE) subtype is selected from (a) painful filling, (b) painful urgency, (c) both painful filling and painful urgency, and (d) neither painful filling nor painful urgency.
5. The method of paragraph 2, wherein the biomarker is selected from A1AT, APOC2, APOA4, ITIH2, and VTNC, and wherein severity of urinary symptoms of Urologic Chronic Pelvic Pain Syndrome (UCPPS) in the subject is scored according to the urinary severity index.
6. The method of paragraph 5, wherein the baseline value for the amount of the biomarker in the sample from the subject is dependent on a characteristic of the subject, wherein the biomarker is selected from A1AT, ITIH2, and VTNC, and wherein the characteristic of the subject is selected from age, Rand Interstitial Cystitis Epidemiology (RICE) subtype, urinary severity index score, and a combination thereof.
7. The method of paragraph 6, wherein a symptom of the Rand Interstitial Cystitis Epidemiology (RICE) subtype is selected from (a) painful filling, (b) painful urgency, (c) both painful filling and painful urgency, and (d) neither painful filling nor painful urgency.
8. The method of paragraph 3, wherein the baseline value for the amount of the biomarker in the sample from the subject is dependent on a characteristic of the subject, wherein the biomarker is selected from A1AT, ITIH2, VTNC, AACT, A1BG, IGHG3, A1AG1, ALBU, and VTDB, and wherein the characteristic of the subject is selected from age, sex, and a combination thereof.
9. The method of paragraph 8, wherein a symptom of the Rand Interstitial Cystitis Epidemiology (RICE) subtype is selected from (a) painful filling, (b) painful urgency, (c) both painful filling and painful urgency, and (d) neither painful filling nor painful urgency.
10. The method of paragraph 2, further comprising using the amount of the biomarker in the sample from the subject to distinguish between (a) a diagnosis of pelvic pain in the subject, and (b) a diagnosis of pelvic pain and beyond in the subject, wherein the biomarker is selected from APOC1 and CO4A.
11. The method of paragraph 10, wherein the biomarker is APOC1, and wherein the amount of the biomarker in the sample from the subject is dependent on a characteristic of the subject, wherein the characteristic of the subject is selected from sex, Rand Interstitial Cystitis Epidemiology (RICE) subtype, and a combination thereof.
12. The method of paragraph 11, wherein a symptom of the Rand Interstitial Cystitis Epidemiology (RICE) subtype is selected from (a) painful filling, (b) painful urgency, (c) both painful filling and painful urgency, and (d) neither painful filling nor painful urgency.
13. The method of paragraph 2, further comprising using the amount of the biomarker in the sample from the subject to assist in diagnosing flare status of Urologic Chronic Pelvic Pain Syndrome (UCPPS) in the subject, wherein the biomarker is GELS.
14. The method of paragraph 2, wherein the amount of the biomarker in the sample from the subject is increased relative to the amount of the biomarker in the reference, wherein the biomarker is selected from C1R and A1AT, and wherein the reference is a reference sample from a control subject, wherein the control subject does not have Urologic Chronic Pelvic Pain Syndrome (UCPPS).
15. The method of paragraph 2, wherein the biomarker is A1AT, and wherein the amount of the biomarker in the sample from the subject is dependent on a characteristic of the subject, wherein the characteristic of the subject is selected from age, sex, urinary severity index score, and a combination thereof.
16. The method of paragraph 2, wherein the biomarker is C1R, and wherein the amount of the biomarker in the sample from the subject is dependent on a characteristic of the subject, wherein the characteristic of the subject is selected from age, sex, and a combination thereof.
17. The method of paragraph 2, wherein the biomarker is selected from APOA1, A1BG, APOE, A1AT, IGHG3, ALBU, VTDB, VTNC, and ITIH2, and wherein the amount of the biomarker in the sample from the subject relative to the amount of the biomarker in the reference determines the subject's Rand Interstitial Cystitis Epidemiology (RICE) subtype.
18. The method of paragraph 17, wherein a symptom of the Rand Interstitial Cystitis Epidemiology (RICE) subtype is selected from (a) both painful filling and painful urgency, and (b) neither painful filling nor painful urgency.
19. The method of paragraph 2, wherein the biomarker is selected from AACT, A1BG, C1R, A1AT, IGHG3, VTDB, VTNC, IC1, and wherein the amount of the biomarker in the sample from the subject relative to the amount of the biomarker in the reference determines the subject's Rand Interstitial Cystitis Epidemiology (RICE) subtype.
20. The method of paragraph 19, wherein a symptom of the Rand Interstitial Cystitis Epidemiology (RICE) subtype is selected from (a) painful urgency, and (b) neither painful filling nor painful urgency.
21. The method of paragraph 2, wherein the biomarker is A1AT, and wherein the amount of the biomarker in the sample from the subject relative to the amount of the biomarker in the reference determines the subject's Rand Interstitial Cystitis Epidemiology (RICE) subtype.
22. The method of paragraph 21, wherein a symptom of the Rand Interstitial Cystitis Epidemiology (RICE) subtype is selected from (a) painful urgency, and (b) both painful filling and painful urgency.
23. The method of paragraph 2, wherein the biomarker is selected from A1AT, A1AG1, and VTNC, and wherein the amount of the biomarker in the sample from the subject relative to the amount of the biomarker in the reference determines the subject's Rand Interstitial Cystitis Epidemiology (RICE) subtype.
24. The method of paragraph 23, wherein a symptom of the Rand Interstitial Cystitis Epidemiology (RICE) subtype is selected from (a) painful filling, and (b) painful urgency.
25. The method of paragraph 2, wherein the amount of the biomarker in the sample from the subject is dependent on a characteristic of the subject, wherein the characteristic of the subject is sex, and wherein the biomarker is selected from IC1, AACT, APOC1, and KLKB1.
26. The method of paragraph 2, wherein the amount of the biomarker in the sample from the subject is dependent on a characteristic of the subject, wherein the characteristic of the subject is age, and wherein the biomarker is selected from A1AG1 and ALBU.
27. The method of paragraph 2, wherein the amount of the biomarker in the sample from the subject is dependent on a characteristic of the subject, wherein the characteristic of the subject is selected from sex, age, and a combination thereof, and wherein the biomarker is selected from A1BG, IGHG3, and VTDB.
28. The method of paragraph 2, wherein the amount of the biomarker in the sample from the subject is dependent on a characteristic of the subject, wherein the characteristic of the subject is selected from age, urinary severity index score, and a combination thereof, and wherein the biomarker is ITIH2.
29. The method of paragraph 2, wherein the amount of the biomarker in the sample from the subject is dependent on a characteristic of the subject, wherein the characteristic of the subject is selected from age, sex, urinary severity index score, and a combination thereof, and wherein the biomarker is selected from A1AT and VTNC.
30. The method of paragraph 1, further comprising administering a treatment for Urologic Chronic Pelvic Pain Syndrome (UCPPS) to the subject.
31. The method of paragraph 1, further comprising administering a treatment for a symptom of Urologic Chronic Pelvic Pain Syndrome (UCPPS) to the subject.
32. The method of paragraph 1, wherein the amount of the biomarker is detected by mass spectrometry, an immunoassay, or a capture and detection assay.
33. The method of paragraph 32, wherein the mass spectrometry comprises operating a mass spectrometer.
34. The method of paragraph 32, wherein the immunoassay comprises performing the immunoassay.
35. The method of paragraph 32, wherein the capture and detection assay comprises performing the capture and detection assay.
36. The method of paragraph 1, wherein the biomarker is an isoform, single-nucleotide polymorphism, or fragment of the protein.
37. The method of paragraph 1, wherein the protein comprises a post-translational modification.
38. The method of paragraph 1, wherein the subject is human.

Various embodiments of the present invention are described in the ensuing examples. The examples are intended to be illustrative and in no way restrictive.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

The invention will be further explained by the following examples, which are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Methods: We collected serum samples from 400 patients who participated in the Multidisciplinary Approach to the study of chronic Pelvic Pain (MAPP) network. We applied multiple reaction monitoring mass spectrometry (MRM-MS) methods for 19 pre-selected targeted proteins (Table 2) that are involved in many diseases and inflammatory processes. The largest categories of study were control vs UCPPS. We also matched patients by pain severity, sex, pelvic pain vs. pelvic pain and beyond (widespread pain), and flare status. These were processed and analyzed.

Results Obtained: Proteins had significant differential expression across seven categories, including age, sex, pelvic pain, RICE subtype, flare status, cohort (control vs. UCPPS), and urinary severity. We also identified interactions between proteins and their overlap across comparison groups. Many markers had overlap between, for example, urinary severity and effect of age. Some markers were seen across three or more comparisons.

Conclusions: The targeted analysis of 19 proteins (Table 2), which are involved in multiple pathways including inflammation, appears to distinguish patients with UCPPS vs. controls. Depending on the peptide it also distinguishes between sex, age, and urinary severity. Understanding the signaling networks perturbed in UCPPS will open new avenues to the identification of novel biomarkers and, equally important, drug targets.

### Example 2

Introduction: We applied three mass spectrometry approaches; de novo discovery, SWATH, and MRM to identify UCPPS-associated urinary biomarkers in serum. De novo discovery was performed using the Thermo Orbitrap ELITE MS instrument. The Orbitrap was operated in a data-dependent mode. Without being bound by theory, this method allows for new post translational modifications (PTMs) and sites of modification to be discovered. Without limitation and without being bound by theory, MS-based quantitation by label free and chemical-tags like iTRAQ (isobaric tag for relative and absolute quantification) and TMT (tandem mass tag) methods can be employed here, both of which can capture and quantify Cys-modified and other PTM forms. Without being bound by theory, SWATH is based on data-independent acquisition of all observable peptides that are present in a sample. Without being bound by theory, this method is based on peptide fragmentation databases that are unique to a particular type. Without being bound by theory, MRM selects peptides of interest and can quantify their levels.

Approach: Separate aliquots of serum specimens of UCPPS patients and matched controls (female and male, n=300) were collected through the MAPP Network. Specimens were analyzed in a blind manner. The pH was adjusted and protein concentration was determined by Bradford assay. Equal amounts of proteins were processed in Vivaspin 6 spin-filters. Validation of actual modified sites were carried out as needed.

### Example 3

Determination of UCPPS-associated proteome composition and relative changes using de novo discovery methods. Discovery-based analysis was performed in which samples were quantitatively compared using gel based and liquid chromatographic (LC) MS methods. Highly abundant proteins (for serum the top 14 abundant proteins) were depleted using affinity chromatography. Fractions were neutralized and digested prior to analysis by MS. Quality control included tracking efficiency of depletion using MRM assays for each of the depleted proteins and ensuring full digestion by 1DE and MS on 10% of the sample. Samples were randomized to eliminate any potential bias, and subsets were repeated.

### Example 4

Determination of phosphorylation and other protein modifications. Gel-based methods (differential in-gel electrophoresis (DIGE) 2DE in which proteins are separated under reducing and denaturing conditions first by pH followed by molecular weight) can resolve phosphorylated proteins based on a pI (pH) shift. One common approach to analyze two samples (each labeled with a different Cys-Dye) in which the endogenous sample(s) are run simultaneously with an equivalent sample that had been dephosphorylated. This allows for the unambiguous identification of phosphorylated proteins, based on a corresponding pI shift difference observed in the same gel between the two different samples.

Direct MS detection. Isolated proteins were tryptic digested and subjected to phospho-peptide enrichment, using TiO2 enrichment for phosphor-Ser/Thr using TiOx affinity chromatography or immuno-enrichment for phospho-Tyr, and analyzed on the Orbitrap ELITE MS instrument. Phospho-Tyr can be isolated using a number of effective anti-phospho-Tyr antibodies using affinity chromatography. For global analysis of serum, without limitation we used SWATH, so as without being bound by theory to ensure elimination of quantitation biases that can reduce the ability to quantify low abundant sites (peptides). Individual proteins, protein complexes or complex subproteomes are analyzed this way.

Data collection, analysis, and interpretation. MS data was searched against the NCBI and or Uniprot protein databases. For the ESI instruments, we either used Sorcerer™ Sequest rescoring, with PeptideProphet and ProteinProphet algorithms, or utilized the PASS platform, which combines data obtained from 3 different search engines, X!Tandem, OSMA and Mascott, maximizing MS spectra assignment for the discovery based analysis. Data were filtered for high confidence identifications (<1% FDR). PTMs were analyzed using a score to help assign probability of amino assignment. For SWATH, data dependent acquisition files were imported into Peakview at a 5% protein FDR to generate the peptide spectral library. SWATH acquisition files were then searched against the peptide spectral library using the SWATH processing software within Peakview. Peptides were extracted at 1% FDR in the SWATH processing software using minimum of 5 transitions per peptide. Quantification and statistical analysis were performed in Markerview. We investigated what proteins are differentially expressed in UCPPS patient serum specimens compared to controls. We used a two-sided two-sample T-Test with false discovery rate (FDR) of 0.05. For each test the individual test alpha was 0.00096.

Many variations and alternative elements have been disclosed in embodiments of the present invention. Still further variations and alternate elements will be apparent to one of skill in the art. Various embodiments of the invention can specifically include or exclude any of these variations or elements.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method for determining status of Urologic Chronic Pelvic Pain Syndrome (UCPPS) in a subject, comprising:
obtaining a sample from the subject,
wherein the subject has Urologic Chronic Pelvic Pain Syndrome (UCPPS), and
wherein the sample is selected from blood, serum, plasma, urine, and a combination thereof;
detecting an amount of at least one biomarker in the sample, wherein the biomarker is a protein selected from A1AT, APOC2, APOA4, ITIH2, VTNC, APOC1, CO4A, GEL, IC1, AACT, APOA1, A1BG, APOE, C1R, IGHG3, A1AG1, ALBU, VTDB, KLKB1, and a combination thereof; and
comparing the amount of the at least one biomarker in the sample from the subject to an amount of the at least one biomarker in a reference to determine the status of Urologic Chronic Pelvic Pain Syndrome (UCPPS) in a subject.

2. The method of claim 1, wherein the reference is selected from:
(i) a baseline value for the amount of the biomarker, wherein the baseline value is from at least one sample obtained from the subject at an earlier point in time;
(ii) a reference sample from a control subject, wherein the control subject does not have Urologic Chronic Pelvic Pain Syndrome (UCPPS);
(iii) a reference sample from a control subject, wherein the control subject has Urologic Chronic Pelvic Pain Syndrome (UCPPS);
(iv) a range of reference values for the amount of the biomarker, wherein the range of reference values is from at least one reference sample obtained from at least one healthy subject;
(v) a range of reference values for the amount of the biomarker, wherein the range of reference values is from at least one sample obtained from the subject at an earlier point in time; and
(vi) a range of reference values for the amount of the biomarker, wherein the range of reference values is from at least one reference sample obtained from at least one reference subject, wherein the reference subject has Urologic Chronic Pelvic Pain Syndrome (UCPPS).

3. The method of claim 2, wherein an increase in the amount of the at least one biomarker in the sample from the subject relative to the amount of the at least one biomarker in the reference is used to determine a Rand Interstitial Cystitis Epidemiology (RICE) subtype in the subject,
wherein the biomarker is selected from A1AT, ITIH2, VTNC, APOC1, IC1, AACT, APOA1, A1BG, APOE, C1R, IGHG3, A1AG1, ALBU, VTDB, and KLKB1.

4. (canceled)

5. The method of claim 2, wherein the biomarker is selected from A1AT, APOC2, APOA4, ITIH2, and VTNC, and
wherein severity of urinary symptoms of Urologic Chronic Pelvic Pain Syndrome (UCPPS) in the subject is scored according to the urinary severity index.

6. The method of claim 5, wherein the baseline value for the amount of the biomarker in the sample from the subject is dependent on a characteristic of the subject,
wherein the biomarker is selected from A1AT, ITIH2, and VTNC, and
wherein the characteristic of the subject is selected from age, Rand Interstitial Cystitis Epidemiology (RICE) subtype, urinary severity index score, and a combination thereof.

7. (canceled)

8. The method of claim 3, wherein the baseline value for the amount of the biomarker in the sample from the subject is dependent on a characteristic of the subject,
wherein the biomarker is selected from A1AT, ITIH2, VTNC, AACT, A1BG, IGHG3, A1AG1, ALBU, and VTDB, and
wherein the characteristic of the subject is selected from age, sex, and a combination thereof.

9. (canceled)

10. The method of claim 2, further comprising using the amount of the biomarker in the sample from the subject to distinguish between (a) a diagnosis of pelvic pain in the subject, and (b) a diagnosis of pelvic pain and beyond in the subject,
wherein the biomarker is selected from APOC1 and CO4A.

11. The method of claim 10, wherein the biomarker is APOC1, and
wherein the amount of the biomarker in the sample from the subject is dependent on a characteristic of the subject,
wherein the characteristic of the subject is selected from sex, Rand Interstitial Cystitis Epidemiology (RICE) subtype, and a combination thereof.

12. (canceled)

13. The method of claim 2, further comprising using the amount of the biomarker in the sample from the subject to assist in diagnosing flare status of Urologic Chronic Pelvic Pain Syndrome (UCPPS) in the subject,
wherein the biomarker is GELS.

14. The method of claim 2, wherein the amount of the biomarker in the sample from the subject is increased relative to the amount of the biomarker in the reference,
wherein the biomarker is selected from C1R and A1AT, and
wherein the reference is a reference sample from a control subject, wherein the control subject does not have Urologic Chronic Pelvic Pain Syndrome (UCPPS).

15. The method of claim 2, wherein the biomarker is A1AT, and
wherein the amount of the biomarker in the sample from the subject is dependent on a characteristic of the subject,
wherein the characteristic of the subject is selected from age, sex, urinary severity index score, and a combination thereof.

16. The method of claim 2, wherein the biomarker is C1R, and
wherein the amount of the biomarker in the sample from the subject is dependent on a characteristic of the subject,
wherein the characteristic of the subject is selected from age, sex, and a combination thereof.

17. The method of claim 2, wherein the biomarker is selected from APOA1, A1BG, APOE, A1AT, IGHG3, ALBU, VTDB, VTNC, and ITIH2, and
wherein the amount of the biomarker in the sample from the subject relative to the amount of the biomarker in the reference determines the subject's Rand Interstitial Cystitis Epidemiology (RICE) subtype.

18. (canceled)

19. The method of claim 2, wherein the biomarker is selected from AACT, A1BG, C1R, A1AT, IGHG3, VTDB, VTNC, IC1, and
wherein the amount of the biomarker in the sample from the subject relative to the amount of the biomarker in the reference determines the subject's Rand Interstitial Cystitis Epidemiology (RICE) subtype.

20. (canceled)

21. The method of claim 2, wherein the biomarker is A1AT, and
wherein the amount of the biomarker in the sample from the subject relative to the amount of the biomarker in the reference determines the subject's Rand Interstitial Cystitis Epidemiology (RICE) subtype.

22. (canceled)

23. The method of claim 2, wherein the biomarker is selected from A1AT, A1AG1, and VTNC, and
wherein the amount of the biomarker in the sample from the subject relative to the amount of the biomarker in the reference determines the subject's Rand Interstitial Cystitis Epidemiology (RICE) subtype.

24. (canceled)

25. The method of claim 2, wherein the amount of the biomarker in the sample from the subject is dependent on a characteristic of the subject,
wherein the characteristic of the subject is sex, and
wherein the biomarker is selected from IC1, AACT, APOC1, and KLKB1.

26. The method of claim 2, wherein the amount of the biomarker in the sample from the subject is dependent on a characteristic of the subject,
wherein the characteristic of the subject is age, and
wherein the biomarker is selected from A1AG1 and ALBU.

27. The method of claim 2, wherein the amount of the biomarker in the sample from the subject is dependent on a characteristic of the subject,
wherein the characteristic of the subject is selected from sex, age, and a combination thereof, and
wherein the biomarker is selected from A1BG, IGHG3, and VTDB.

28. The method of claim 2, wherein the amount of the biomarker in the sample from the subject is dependent on a characteristic of the subject,
wherein the characteristic of the subject is selected from age, urinary severity index score, and a combination thereof, and
wherein the biomarker is ITIH2.

29. The method of claim 2, wherein the amount of the biomarker in the sample from the subject is dependent on a characteristic of the subject,
wherein the characteristic of the subject is selected from age, sex, urinary severity index score, and a combination thereof, and
wherein the biomarker is selected from A1AT and VTNC.

30. The method of claim 1, further comprising administering a treatment for Urologic Chronic Pelvic Pain Syndrome (UCPPS) to the subject.

31. The method of claim 1, further comprising administering a treatment for a symptom of Urologic Chronic Pelvic Pain Syndrome (UCPPS) to the subject.

32. The method of claim 1, wherein the amount of the biomarker is detected by mass spectrometry, an immunoassay, or a capture and detection assay.

33. The method of claim 32, wherein
the mass spectrometry comprises operating a mass spectrometer,
the immunoassay comprises performing the immunoassay, and
the capture and detection assay comprises performing the capture and detection assay.

34. (canceled)

35. (canceled)

36. The method of claim 1, wherein the biomarker is an isoform, single-nucleotide polymorphism, or fragment of the protein.

37. The method of claim 1, wherein the protein comprises a post-translational modification.

38. The method of claim 1, wherein the subject is human.
